(19) United States
(12) Patent Application Publication
(10) Pub. No.: US 2015/0359911 A1
(43) Pub. Date: Dec. 17, 2015

(54) Aptamer Specific to Integrin alpha-v-beta-3 and Use Thereof

(71) Applicants: POSTECH ACADEMY-INDUSTRY FOUNDATION, Pohang (KR); YONSEI UNIVERSITY, UNIVERSITY-INDUSTRY FOUNDATION (UIF), Seoul (KR)

(72) Inventors: Jung Hwan LEE, Pohang (KR); Seungjoo HAAM, Seoul (KR); Yong-min HUH, Seoul (KR); Young Chan CHAE, Pohang (KR); Sung Ho RYU, Pohang (KR); Eun-kyung LIM, Suwon (KR); Jong In KIM, Pohang (KR)

(21) Appl. No.: 14/418,266

(22) PCT Filed: Jul. 31, 2013
(86) PCT No.: PCT/KR2013/006889

§ 371 Date: Jan. 29, 2015

(30) Foreign Application Priority Data
Jul. 31, 2012 (KR) 10-2012-0084069

(51) Int. Cl. A61K 49/18; G01N 33/574

(52) U.S. Cl. CPC A61K 49/1866; G01N 33/57492; G01N 2333/70557

(57) ABSTRACT

The present invention relates to DNA aptamer specifically binding to integrin αᵥβ₃, and a composition for diagnosis of cancer or cancer metastasis comprising the same as an active ingredient. And, the present invention relates to a composition for imaging tumor regions comprising the aptamer, and a contrast medium comprising the same.

### TECHNICAL FIELD

The present invention relates to DNA aptamer specifically binding to integrin αᵥβ₃, and a composition for diagnosis of cancer or cancer metastasis comprising the same as an active ingredient. And, the present invention relates to a composition for imaging tumor regions comprising the aptamer, and a contrast medium comprising the same.

### BACKGROUND ART

Integrin is a cell surface receptor that controls important physiological functions of cells such as cell adhesion, migration, differentiation, proliferation and the like. Integrin is a heterodimer consisting of non-covalently bonded α and β subunits, and the α and β subunits make a pair to constitute 22 kinds of integrin families Although integrin predominantly binds to extracellular matrix protein such as vibronectin, fibronectin, collagen, laminin, vWF, fibrinogen and the like, ligand specificity is varied according to the kinds of integrin, and one kind of integrin may simultaneously bind to various ligands.

Among them, integrin αᵥβ₃ is known to be expressed in aggressive tumor cells including skin cancer, prostate cancer, breast cancer, uterine cervical cancer, colorectal cancer, lung cancer, gallbladder cancer, pancreatic cancer, and stomach cancer, and to control the growth, survival and penetration of adhesion-dependent tumor cells, thus increasing malignancy of various human tumors. Recently, it was also found out that integrin αᵥβ₃ controls cell signal transduction and increases tumor growth and metastasis as adhesion-independent medium (David A Cheresh et al., Nature Medicine 2009, 15 (10): 1163).

More specifically, integrin αᵥβ₃ converts benign radial tumor growth into malignant vertical growth phase in skin cancer, and mediates bone metastasis through increased tumor cell adhesion in prostate cancer and breast cancer. The expression of integrin αᵥβ₃ in uterine cervical cancer correlates to the progress of disease and short survival period, and the expression of integrin αᵥβ₃ in pancreatic ductal adenocarcinoma is observed in about 58% of human tumors and relates to increased lymph node metastasis.

As such, since integrin is specifically expressed in various cancer cells and involved in cancer progress and metastasis, a possibility of developing integrin as a diagnostic marker and treatment target of cancer or cancer metastasis has been suggested.

As an example, Brooks et. al (1994) reported that integrin protein specifically expressed only in vascular endothelial cells of cancer tissues is a biochemical marker of new blood vessels, and Gasparini et. al (1998) confirmed this in breast cancer tissues. Recently, attempts are being made to find out specific markers according to organs or tumor using a peptide library using bacteriophage, and Pasqualini et al. (1997) has reported that Arg-Gly-Asp (RGD) peptide-containing phage specifically binds to integrin. Such RGD peptide is being developed as integrin antagonist.

And, U.S. Pat. No. 6,171,588 provides monoclonal antibody specifically binding to integrin αᵥβ₃ for use in detection or treatment of tumor regions, and US Laid-open Patent No. 20090263320 provides a cancer diagnostic reagent using a peptide based compound specifically binding to integrin αᵥβ₃.

However, antibody or peptide specifically recognizing integrin has problems in that it is difficult to prepare because of the large molecule size, is not easy to modify, and cannot be stored or transported at room temperature and thus has low stability. And, when injected into the body, immune rejection response may occur.

### DISCLOSURE

### Technical Problem

Thus, the inventors studied in order to solve the existing problems and provide novel material that specifically recognize and binds to integrin αᵥβ₃, and discovered DNA based aptamer that specifically binds to integrin αᵥβ₃ with high affinity.

The aptamer of the present invention has advantages in that it has superior stability and sensitivity to the existing protein based agents, is easy to prepare due to the small size, can be produced with low cost within a short time by chemical synthesis, and can be variously modified to increase binding capacity.

And, since the aptamer of the present invention detects integrin αᵥβ₃ with high stability and sensitivity, it may be usefully used for diagnosis of all kinds of cancer relating to integrin αᵥβ₃ and metastasis thereof. Practically, the inventors confirmed cancer targetability of the newly discovered aptamer, succeeded in preparing magnetic nanoparticles conjugated with the aptamer and imaging cancer cells with MRI, and completed the invention.

### Technical Solution

It is an object of the invention to provide aptamer specifically binding to integrin αᵥβ₃.

It is another object of the invention to provide a composition for diagnosis of cancer or cancer metastasis comprising the aptamer as an active ingredient.

It is still another object of the invention to provide a method for providing information on the diagnosis of cancer or cancer metastasis using the aptamer.

It is still another object of the invention to provide a method of diagnosis of cancer or cancer metastasis using the aptamer.

It is still another object of the invention to provide a composition for imaging tumor regions comprising the aptamer as an active ingredient.

It is still another object of the invention to provide a method of imaging tumor regions comprising the step of administering the aptamer to a subject.

It is still another object of the invention to provide a nanoparticle contrast media comprising the composition for imaging tumor regions.

### Advantageous Effects

The present invention provide aptamer with high binding capacity and selectivity to integrin αᵥβ₃, and the aptamer may be usefully used for the diagnosis of all kinds of cancer relating to integrin αᵥβ₃ and metastasis thereof.

### DESCRIPTION OF DRAWINGS

FIG. 1 shows the results of confirming an ester (—COO—) structure that is formed when preparing P80-triCOOH through infrared spectrum.

FIG. 2 shows the process of preparing integrin αᵥβ₃ aptamer conjugated magnetic nanoparticles.

FIG. 3 (a) shows the result of measuring the size of the prepared integrin αᵥβ₃ aptamer conjugated magnetic nanoparticles by dynamic laser light scattering, (b) shows the result of confirming the shape of the particles by transmission electron microscope, (c) shows the result of confirming super paramagnetism using a vibrating sample magnetometer, and (d) shows the result of measuring the content of magnetic nanoparticles by a thermal analyzer.

FIG. 4 (a) confirms that when integrin αᵥβ₃ aptamer is applied for MR image experiment, at 1.5T, as the concentration of magnetic nanoparticles increases, remarkably dark MR contrast appears, and (b) confirms that as the concentration increases, r2 (T2 relaxvity coefficients) increases.

FIG. 5 shows the results of confirming that integrin αᵥβ₃ aptamer conjugated magnetic nanoparticles (Apt_{αvβ3}-MNPs) and cRGD conjugated magnetic nanoparticles (cRGD-MNPs) have stabilities under serum containing buffer conditions according to concentrations.

FIG. 6 shows that neither integrin αᵥβ₃ aptamer conjugated magnetic nanoparticles (Apt_{αvβ3}-MNPs) and cRGD conjugated magnetic nanoparticles (cRGD-MNPs) exhibit cytotoxicity even at high concentrations.

FIG. 7 (a) shows the results of confirming the image contrast effects over time when PAE/KDR (integrin αᵥβ₃ overexpressing, test group) cells and A431 (integrin αᵥβ₃ low expressing, control) cells are respectively treated with Apt_{αvβ3}-MNPs and cRGD-MNPs, and (b) shows a graph of signal increase rate plotted by measuring the MR signal intensity (R2) from the image results of FIG. 7 (a), based on the R2 value of non-treated cells.

FIG. 8 (a) shows the results of confirming image contrast effects over time in cancer tissue after injecting Apt_{αvβ3}-MNPs and cRGD-MNPs respectively in a cancer animal model, and (b) shows a graph of MR signal intensity plotted from the image results of FIG. 8 (a).

FIG. 9 shows the results of measuring the amounts of Apt_{αvβ3}-MNPs and cRGD-MNPs accumulated in each organ extracted from each animal by plasma-atomic emission spectrometry, after injecting Apt_{αvβ3}-MNPs and cRGD-MNPs respectively in cancer animal models and confirming 24 hour images.

### BEST MODE

The present invention provides DNA aptamer specifically binding to integrin αᵥβ₃, a composition for diagnosis of cancer or cancer metastasis comprising the same as an active ingredient, and a method of diagnosis of cancer or cancer metastasis using the same. The present invention also provides a composition for imaging tumor regions comprising the aptamer, and a contrast media comprising the same.

According to one embodiment, the present invention relates to aptamer specifically binding to integrin αᵥβ₃.

The integrin αᵥβ₃ may be derived from mammals, preferably human being.

The aptamer may comprise modified bases, it may have a total base length of 25 to 100, preferably 30 to 80, more preferably 35 to 50 including modified bases, and it specifically binds to integrin αᵥβ₃. In the aptamer of the present invention, unless otherwise described, bases other than the modified bases are selected from the group consisting of A, G, C, T, and deoxy forms thereof (for example, 2′-deoxy form).

The modified base refers to a modified form of dU (deoxyuracil) that is substituted at the 5-position with a hydrophobic functional group, and it may be used instead of base ‘T’. The hydrophobic functional group may be at least one selected from the group consisting of a benzyl group, a naphthyl group, a pyrrole benzyl group, and tryptophan, and the like, and preferably a benzyl group. As such, since the 5-position of dU base is substituted with a hydrophobic functional group and modified, affinity with integrin αᵥβ₃ remarkably increases compared to non-modified case.

The number of modified bases in the aptamer of the present invention may be 5 to 20, preferably 10 to 17.

In a specific embodiment, the aptamer including at least one base sequence selected from the group consisting of SEQ ID NO: 1 to SEQ ID NO: 56 (in the base sequence ‘n’ is the modified base or ‘T’) may have a total base length of 25 to 100, preferably 30 to 80, more preferably 35 to 50.

In a specific embodiment, the aptamer may consist only of at least one base sequence selected from the group consisting of SEQ ID NO: 1 to SEQ ID NO: 56, or it may further comprise a base sequence consisting of 3 to 25, specifically 5 to 20 bases at the 5′terminal, 3′terminal or both terminals of the base sequence, thus having a total base length of 30 to 120, 35 to 100, or 45 to 90. The base sequence further included in the 5′terminal, 3′terminal or both terminals may be selected from the group consisting of SEQ ID NO: 57 to SEQ ID NO: 60. For example, the aptamer may have TCAGCCGCCAGCCAGTTC (SEQ ID NO: 57) at the 5′terminal and GACCAGAGCACCACAGAG (SEQ ID NO: 58) at the 3′terminal of at least one base sequence selected from the group consisting of SEQ ID NO: 1 to SEQ ID NO: 56, or it may have AGTTC (SEQ ID NO: 59) at the 5′terminal and GACCA (SEQ ID NO: 60) at the 3′terminal, but is not limited thereto.

In a specific embodiment, the aptamer of the invention may have a base sequence of the following SEQ ID NO: 61 or SEQ ID NO: 62:

5′-TCAGCCGCCAGCCAGTTC-[N]-GACCAGAGCACCACAGAG-3′ (SEQ ID NO: 61)

5′-AGTTC-[N]-GACCA-3′ (SEQ ID NO: 62),

(in the base sequences, N is a variable core sequence of the aptamer, and consists of 25 to 100, preferably 30 to 80, more preferably 35 to 50 bases, and each base is independently selected from the group consisting of A, C, G, T, deoxy forms thereof, and modified bases of dU (deoxyuracil) that are substituted at the 5′-position with a hydrophobic functional group (for example, at least one selected from the group consisting of a benzyl group, a naphthyl group, a pyrrole benzyl group, and tryptophan)).

As explained above, the N may be selected from the group consisting of SEQ ID NO: 1 to SEQ ID NO: 56.

In the base sequence described herein and in the attached base sequence, ‘n’, unless otherwise described, refers to ‘T’ or a modified form of dU (deoxyuracil) that is substituted at the 5′position with a hydrophobic functional group, preferably a modified form of dU (deoxyuracil) that is substituted at the 5-position with a hydrophobic functional group. The hydrophobic functional group is at least one selected from the group consisting of, a benzyl group, a naphthyl group, a pyrrole benzyl group, tryptophan, and the like, preferably a benzyl group.

And, the aptamer may be modified at the 5′terminal, 3′terminal or both terminals so as to improve serum stability. The aptamer may be modified by binding of at least one selected from the group consisting of PEG (polyethylene glycol), idT (inverted deoxythymidine), LNA (Locked Nucleic Acid), 2′-methoxy nucleoside, 2′-amino nucleoside, 2′F-nucleoside, amine linker, thiol linker, and cholesterol and the like to the 5′terminal, 3′terminal or both terminals. In a preferable embodiment, the aptamer may include PEG (polyethylene glycol; for example, molecular weight 500-50,000 Da) attached to the 5′terminal, idT (inverted deoxythymidine) attached to the 3′terminal, or PEG (for example, molecular weight 500-50,000 Da) attached to the 5′terminal and idT (inverted deoxythymidine) attached to the 3′terminal.

The ‘idT (inverted deoxythymidine)’ is one of the molecules used to prevent decomposition of aptamer with low nuclease resistance by nuclease. Although nucleic acid unit binds 3′-OH of the previous unit with 5′-OH of the next unit to form a chain, idT binds 3′-OH of the previous unit with 3′-OH of the next unit to cause artificial change so that 5′-OH is exposed instead of 3′-OH, thereby inhibiting decomposition by 3′ exonuclease, which is a kind of nuclease.

Since overexpression of integrin αᵥβ₃ is observed in patients with various solid cancers and metastasis, the aptamer of the present invention can be used as a composition for diagnosis of cancer or cancer metastasis.

Thus, according to another embodiment, the present invention relates to a composition for diagnosis of cancer or cancer metastasis comprising the integrin αᵥβ₃ aptamer specifically binding to integrin αᵥβ₃ as an active ingredient.

According to yet another embodiment, the present invention relates to a method for providing information on the diagnosis of cancer or cancer metastasis using the integrin αᵥβ₃ aptamer specifically binding to integrin αᵥβ₃.

Preferably, the method for providing information on the diagnosis of cancer or cancer metastasis comprises the steps of reacting a biological sample of a patient with the aptamer of the present invention, and measuring binding degree of aptamer in the biological sample,

wherein if the binding degree of aptamer in the biological sample of the patient is higher than that in a normal sample, the patient is determined as a cancer patient. Thus, the method may further comprise the step of measuring binding degree of aptamer in a normal sample.

The patient may be mammals including human, preferably rodents or human, and it means a subject in which cancer development or cancer metastasis is to be judged.

The cancer, on the diagnosis of which information can be provided by the method, may include all kinds of cancer relating to integrin αᵥβ₃, and for example, it may be at least one selected from the group consisting of skin cancer, prostate cancer, breast cancer, uterine cervical cancer, colorectal cancer, lung cancer, gallbladder cancer, pancreatic cancer, stomach cancer, and the like.

The normal sample may be obtained from mammals including human, preferably from rodents or human, and it means a biological sample obtained from a subject without development and metastasis of cancer, on the diagnosis of which information is to be provided, for example, cancer selected from the group consisting of skin cancer, prostate cancer, breast cancer, uterine cervical cancer, colorectal cancer, lung cancer, gallbladder cancer, pancreatic cancer, stomach cancer, and the like.

The biological sample may be a mammal body except human, cells, tissues, blood, saliva and the like, separated from mammals including human.

The step of measuring binding degree of aptamer in the biological sample may be conducted by measuring technology of DNA aptamer binding commonly used in related technological field, and for example, the end of the aptamer may be labeled with fluorescent or radioactive material or bound with biotin to measure the intensity of fluorescence or radioactivity, or it may be imaged and observed, but is not limited thereto.

According to one specific embodiment, among the aptamers, one pair of aptamers that have different binding regions with integrin αᵥβ₃ and do not hinder binding each other are selected, one is fixed on a substrate (capture aptamer), and the other (detection aptamer) is labeled with a detectable label at the end, and the intensity is measured, thereby measuring whether or not integrin αᵥβ₃ exists in the sample or whether or not integrin αᵥβ₃ is overexpressed.

The detectable label may be fluorescent or radioactive label (or bound with material that can react with fluorescent or radioactive substance), and for example, it may include colorimetric enzyme (for example, peroxidase, alkaline, phosphatase), radioisotope (for example: ¹²⁴I, ¹²⁵I, ¹¹¹In, ^{99m}Tc, ³²P, ³⁵S), chromophore, FITC, RITC, fluorescent protein (GFP (Green Fluorescent Protein); EGFP (Enhanced Green Fluorescent Protein), RFP (Red Fluorescent Protein); DsRed (Discosoma sp. red fluorescent protein); CFP (Cyan Fluorescent Protein), CGFP (Cyan Green Fluorescent Protein), YFP (Yellow Fluorescent Protein), Cy3, Cy5 and Cy7.5, and the like, but is not limited thereto.

As such, when the existence of integrin αᵥβ₃ in the sample or overexpression of integrin αᵥβ₃ is confirmed using aptamer, remarkably excellent sensitivity is shown compared to detection using the existing antibodies.

Moreover, since the aptamer of the present invention specifically binds to integrin αᵥβ₃, it may be usefully used for imaging of tumor regions.

Thus, according to yet another embodiment, the present invention relates to a composition for imaging tumor regions comprising the aptamer as an active ingredient.

According to yet another embodiment, the present invention relates to a method for imaging tumor regions comprising the step of administering the aptamer to a subject.

The imaging and diagnosis of tumor disease, although not limited hereto, may be used for monitoring of progress, treatment progress, response to therapeutic agent, and the like, as well as for the first medical examination of tumor disease. The aptamer may be linked (for example, covalently bonded or crosslinked) to a detectable label so as to facilitate confirmation of binding, detection and quantification.

Preferably, the detectable label for imaging tumor regions may include radioisotope, fluorophore, quantum dot, and magnetic particles, for example, super paramagnetic particles or ultrasuper paramagnetic particles, and the like, but is not limited thereto.

Preferably, the aptamer of the present invention may be conjugated with nanoparticles and provided as a nanoparticle contrast media for imaging of tumor regions.

Thus, according to yet another embodiment, the present invention relates to a nanoparticle contrast media comprising the composition for imaging tumor regions containing the aptamer of the present invention. Wherein, the aptamer of the present invention specifically binds to integrin αᵥβ₃ and functions as a target ligand for targeting tumor regions, thus enabling rapid and exact diagnosis of tumor regions by active targeting.

As used herein, a “contrast media” refers to an agent used to artificially make difference in contrast and present it as an image so that organs, blood vessels or tissues and the like may be seen better for diagnosis. The contrast media increases visibility and contrast of the surface to be examined, thereby determining the existence of disease or damage, and the degree thereof.

A contrast media should have excellent biocompatibility and biodegradability, and it requires to have excellent in vivo stability and high degree of distribution in blood, thus to be continuously accumulated in cancer tissue for a sufficient time. It is confirmed that the aptamer conjugated nanoparticle contrast media of the present invention has very high accumulation efficiency in cancer tissue, and is safe material that does not have in vivo toxicity and does not exhibit abnormal findings, and thus, it is suitable for use as a contrast media.

The contrast media of the present invention may be applied for magnetic resonance imaging (MRI), X-ray imaging technique, and nuclear imaging including PET (positron emission tomography), but is not limited thereto.

Magnetic resonance imaging (MRI) refers to image diagnosis technique for obtaining anatomical, physiological and biochemical information of bodies using relaxation of spin of hydrogen atom in magnetic field. If the nanoparticle contrast media of the present invention is applied for MRI, paramagnetic nanoparticles or superparamagnetic nanoparticles widely used in the art may be used. For example, as the paramagnetic particles, transition metal ions such as Gd, Fe, Mn, and the like may be used, and as the superparamagnetic particles, superparamagnetic iron oxide nanoparticles, and the like may be used.

According to one preferred embodiment, the nanoparticle contrast media of the present invention may have a structure including a core containing magnetic nanoparticles and a shell formed by adding an amphiphilic compounds to the core. Wherein the hydrophobic region including a pyrene structure of the amphiphilic compound may be bonded to the surface of nanoparticles by a chemical bond of π-π bond, and the hydrophilic region of the amphiphilic compound may be distributed at the outermost part of the nanoparticle and stabilize water-insoluble nanoparticles even in water soluble medium, thus maximizing bioavailability.

As synthesis methods of the nanoparticles, coprecipitation, hydrothermal synthesis, microemulsion (oil-in-water or water-in-oil), thermal decomposition and the like may be used, but not limited thereto.

The magnetic nanoparticles may have a diameter of 1 to 1000 nm, more preferably 2 to 100 nm, and metals, magnetic substances, or magnetic alloys having the above diameter may be used. As the metal, although not specifically limited, Pt, Pd, Ag, Cu, or Au, and the like may be used alone or in combinations. As the magnetic substance, although not specifically limited, Co, Mn, Fe, Ni, Gd, Mo, MM′₂O₄, or MxOy (M and M′ respectively independently represents Co, Fe, Ni, Mn, Zn, Gd, or Cr, x and y respectively satisfies the equations “0<x≦3” and “0<y≦5”) may be used alone or in combinations. As the magnetic alloy, although not specifically limited, CoCu, CoPt, FePt, CoSm, NiFe, or NiFeCo and the like may be used alone or in combinations.

And, the magnetic nanoparticles may be bonded with an organic surface stabilizer so as to stabilize a bond with an amphiphilic compound. The bond of the magnetic nanoparticles and the organic surface stabilizer may be made by coordination of the organic surface stabilizer with a precursor of the magnetic nanoparticles and the resulting formation of a complex compound.

The organic surface stabilizer means an organic functional molecule that can stabilize the state and size of the nanoparticles, and for example, it may include a surfactant. As the surfactant, cationic surfactant including alkyl trimethylammonium halide; amphoteric surfactant including saturated or unsaturated fatty acid such as oleic acid, lauric acid, or dodecylic acid, trialkylphosphine or trialkylphosphine oxide such as trioctylphosphine oxide (TOPO), trioctylphosphine (TOP), or tributylphosphine, alkyl amine such as oleic amine, trioctylamine, or octylamine, and alkyl thiol; and anionic surfactant including sodium alkyl sulfate, and sodium alkyl phosphate may be used, but not limited thereto.

And, the amphiphilic compound may distribute nanoparticles in a matrix, or be bonded to the surface of nanoparticles, and chemically bind a target ligand to one end of polymer.

The hydrophobic region of the amphiphilic compound may include a hydrophobic compound to which material including a pyrene structure is bonded. As the hydrophobic compound, saturated fatty acid, unsaturated fatty acid, or hydrophobic polymer and the like may be used alone or in combinations. The saturated fatty acid, although not specifically limited, may include butyric acid, caproic acid, caprylic acid, capric acid, lauric acid (dodecyl acid), myristic acid, palmitic acid, stearic acid, eicosanoic acid, docosanoic acid and the like, or combinations thereof. The unsaturated fatty acid, although not specifically limited, may include oleic acid, linoleic acid, linolenic acid, arachidonic acid, eicosapentanoic acid, docasahexanoic acid, erucic acid, or combinations thereof. The hydrophobic polymer, although not specifically limited, may include polyphosphazene, polylactide, polylactide-co-glycolide, polycaprolactone, polyanhydride, polymalic acid or derivatives thereof, polyalkylcyanoacrylate, polyhydroxybutylate, polycarbonate, polyorthoester, hydrophobic polyamino acid, hydrophobic vinyl polymer, or combinations thereof. The material including a pyrene structure, although not specifically limited, may include pyrene, pyrenebutyric acid, pyrene methylamine, 1-aminopyrene, pyrene-1-boronic acid, organic molecules including a pyrene structure, or combinations thereof.

The hydrophilic region of the amphiphilic compound may include polyalkyleneglycol (PAG), polyetherimide (PEI), polyvinylpyrrolidone (PVP), hydrophilic polyamino acid (PAA), hydrophilic vinyl polymer, hydrophilic acryl polymer or dextran, polysaccharide polymer such as hyaluronic acid, and the like, or combinations thereof.

And, the magnetic nanoparticles according to the present invention may include aptamer introduced in the hydrophilic region to afford targetability to the magnetic nanoparticles. The aptamer has a functional group such as —NH₂, —SH, —COOH, and the like at the 5′- and 3′-terminal, and thus, may be usefully used for binding with the functional group of the binding region of active ingredients. And, functional groups such as carboxylic acid, phosphate, sulfate, an amine group, a hydroxyl group, a thiol group and the like on the surface of the nanoparticles may be modified to facilitate binding of aptamer.

The imaging composition or nanoparticle contrast agent of the present invention may further comprise a radiologically acceptable carrier, wherein the radiologically acceptable carrier may include any carriers and vehicles commonly used in the field of pharmaceuticals, and specifically, it may include ion exchange resin, alumina, aluminum stearate, lecithin, serum protein (for example, human serum albumin), buffer (for example, various phosphate, glycin, sorbic acid, potassium sorbate, partial glyceride mixture of saturated vegetable fatty acid), water, salt or electrolytes (for example, protamine sulfate, disodium hydrogen phosphate, potassium hydrogen phosphate, sodium chloride and zinc salt), colloidal silica, magnesium trisilicate, polyvinylpyrrollidone, cellulose substrate, polyethyleneglycol, sodium carboxymethylcellulose, polyarylate, wax, polyethyleneglycol or wool grease, and the like, but is not limited thereto. And, The imaging composition or nanoparticle contrast agent may further comprise a lubricant, a wetting agent, an emulsifier, a suspending agent or a preservative, and the like, in addition to the above ingredients.

Cancer metastasis is the main cause of cancer related death. Particularly, most cancer patients are often diagnosed after metastasis is progressed, and show high recurrence rate despite of surgery and chemotherapy. Thus, treatment of cancer metastasis is the major target of cancer treatment. Cancer cells should overcome various kinds of stresses and rate-limiting steps so that metastasis grows in the microenvironment of new organ. Since integrin αᵥβ₃ is known to be overexpressed in cancer cells and promote the progression to metastatic and malignant tumor, it may become an important target for diagnosis and treatment of cancer or cancer metastasis.

Some antibodies and peptides to integrin αᵥβ₃ have been suggested as an anticancer agent- or radioisotope-carrier as well as treatment strategy of human cancer including metastasis. However, since these antibodies have low affinity (μM range Kd) and undesirable pharmacokinetic properties (for example, they have low tumor invasion capacity and are rapidly removed), they have a limitation in the application for target delivery, and thus, development of high affinity molecules with improved pharmacokinetic properties and serum stability is becoming important.

Meanwhile, since DNA aptamer can be chemically synthesized, is easily modified for in vivo application, and has excellent tumor tissue invasion capacity, it has various advantages as a cancer targeting molecule. Since natural oligonucleotide is sensitive to hydrolysis by nuclease, the inventors conducted SELEX using modified nucleotide of dU (deoxyuracil) substituted at the 5-position with a hydrophobic functional group of benzyl group, so as to increase binding affinity and decrease slow off-rate while increasing nuclease resistance.

Preferably, the aptamer of the present invention may be useful for diagnosis or molecular level imaging of integrin αᵥβ₃-mediated metastasis.

In the specific examples of the invention, magnetic nanoparticles conjugated with the aptamer of the present invention were prepared with a view to using as an MR contrast agent (Example 2), and it was confirmed that the magnetic nanoparticles have targetability to cancer cells, and when the magnetic nanoparticles were administered in cancer animal models and MR images were observed, it was confirmed that the magnetic nanoparticles are accumulated in cancer tissues and effectively image cancer tissues (Example 3). And, it was also confirmed through in vivo safety evaluation that the aptamer is safe material that does not have in vivo toxicity and does not exhibit abnormal findings (Example 3).

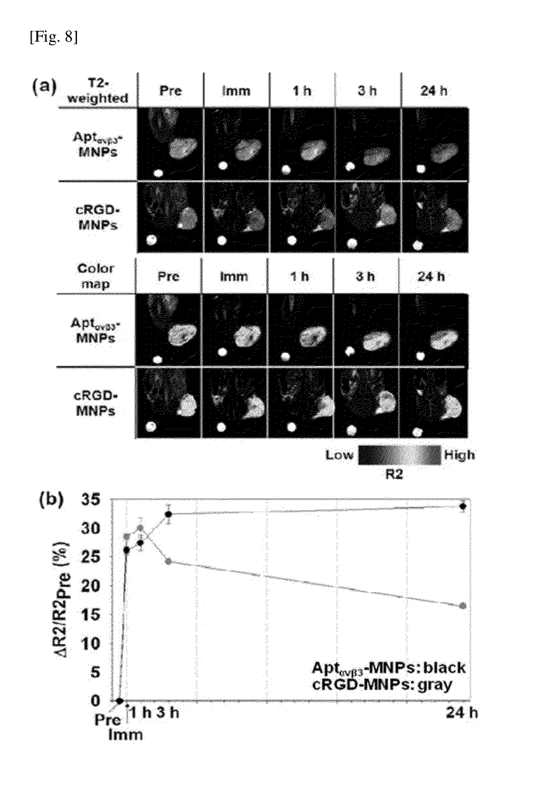

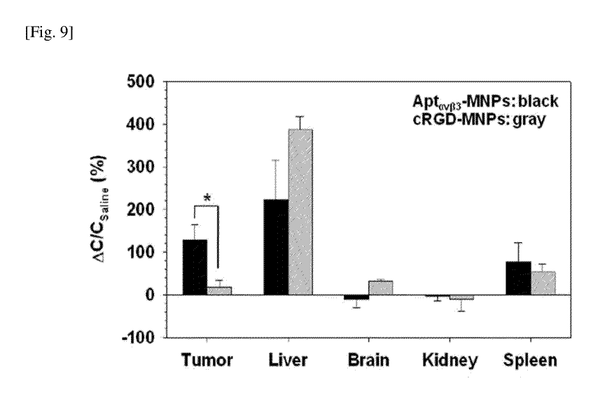

In addition, compared to the case of using magnetic nanoparticles conjugated with cyclo (Arg-Gly-Asp-D-Phe-Lys) (cRGD) peptide, of which integrin αᵥβ₃ targetability has been already well known, in animal models, c-RGD conjugated magnetic nanoparticles exhibited decreased image contrast after 1 hour, while the magnetic nanoparticles conjugated with the aptamer of the present invention exhibited continuous image contrast effect up to 24 hours, exhibited stronger signal intensity, and had more accumulated amount in cancer tissues (FIG. 8 and FIG. 9). Thus, it can be seen that the DNA aptamer of the present invention exhibits superior cancer target imaging effect even to the existing agent cRGD peptide.

Thus, the aptamer of the present invention may be usefully used for measuring the possibility of metastasis and prognosis in cancer patient in vitro and in vivo.

Compared to the existing protein based agents, the DNA aptamer of the present invention exhibits more rapid tumor uptake, more rapid blood removal and more continuous tumor retention, thereby enabling remarkable imaging at a higher ratio of tumor to blood. Thus, the aptamer of the present invention may be usefully used for in vivo imaging of integrin αᵥβ₃ expressing cancer cells particularly in a microenvironment with tumor metastasis.

### MODE FOR INVENTION

Hereinafter, the present invention will be explained in detail with reference to the following examples. However, these examples are only to illustrate the invention, and the scope of the invention is not limited thereto.

### Example 1

### Development of Integrin αᵥβ₃ Aptamer

1.1: Synthesis of Modified Nucleic Acid Library

In order to prepare a single strand modified DNA library required for SELEX, an antisense library with biotin bound at the 5′terminal [5′-Biotin-d(CTC TGT GGT GCT CTG GTC-(N×40)-GAA CTG GCT GGC GGC TGA-3′; (SEQ ID NO: 64)] was synthesized.

With the synthesized antisense, 20 μM 5′ primer (TCA GCC GCC AGC CAG TTC; SEQ ID NO: 65), 0.5 mM dNTP (ATP, GTP, CTP, BzdUTP), 0.25 U/μl KOD XL(KOD XL DNA polymerase, Novagen), 10× extension buffer (1.2M Tris-HCl pH7.8, 100 mM KCl, 60 mM (NH₄)₂SO₄, 70 mM MgSO₄, 1% Triton X-100, 1 mg/ml BSA), incubation was conducted at 70° C. for 1 hours to prepare double strand DNA.

It was eluted using 20 mM NaOH, and then, neutralized with an 80 mM HCl solution to prepare a single strand modified DNA library. The prepared DNA library was concentrated using Amicon ultra-15 (Millipore), and then, quantified with UV spectrophotometer.

1.2: Finding of Integrin αᵥβ₃ Aptamer by SELEX

In order to select DNA aptamer binding to integrin αᵥβ₃ (R&D systems, 3050-AV), SELEX technique was used.

(1) Tagging of Integrin αᵥβ₃:

Integrin αᵥβ₃, which is non-tag protein, was biotinylated with EZ-Link NHS-PEG4-Biotin (Thermo scientific), and then, seeded for SELEX.

(2) Binding with Integrin αᵥβ₃:

First, 1 nmole of the above synthesized library was put in selection buffer (200 mM HEPES, 510 mM NaCl, 25 mM KCl, 25 mM MgCl₂), reacted at 95° C., 70° C., 48° C., 37° C. respectively for 5 minutes, and then, for negative selection, mixed with 10 μL of 10× protein competition buffer (10 μM prothrombin, 10 μM casein, 0.1% (w/v) HSA (human serum albumin, SIGMA), added to supernatant-free Dynabeads® MyOne™ Streptavidin C1 (SA bead) (50% (v/v) slurry, 10 mg/ml Invitrogen) and reacted at 37° C. for 10 minutes.

After the negative selection, only supernatant was taken and transferred to a separate tube, and then, reacted with biotinylated integrin αᵥβ₃ bound Dynal MyOne SA beads at 37° C. for 1 hours. Dynal MyOne SA beads bound with DNA and integrin αᵥβ₃ complex were washed 5 times with 100 μL of selection buffer (200 mM HEPES, 510 mM NaCl, 25 mM KCl, 25 mM MgCl₂). At 5^{th} washing, they were transferred to a new plate and washed. And, 85 μL of a 2 mM NaOH solution was added to elute target-binding library, which was then neutralized with 20 μL of an 8 mM HCl solution.

(3) Amplification:

The target binding library DNA was amplified using QPCR (quantitative PCR, IQ5 multicolor real time PCR detection system, Bio-rad). Each 5 uM (5×QPCR master Mix, Novagen) of the 5′ primer (TCA GCC GCC AGC CAG TTC; SEQ ID NO: 65) and 3′ primer (Biotin-CTC TGT GGT GCT CTG GTC; SEQ ID NO: 66) previously used for preparation of library, 0.075 U/ul KOD (Novagen), 1 mM dNTP (Roche Applied science), and 25 mM MgCl₂, 5×SYBR green I (Invitrogen) were mixed to a total volume of 125 μL, and 1 cycle under conditions of 96° C. 15 seconds, 55° C. 10 seconds, 68° C. 30 minutes, and 30 cycles under conditions of 96° C. 15 seconds, 72° C. 1 minute were repeated to prepare a double strand library.

(4) Preparation of eDNA:

eDNA means aptamer produced using a DNA template and polymerase. The DNA library prepared through QPCR was mixed with 25 μL Myone SA bead (Invitrogen) at room temperature for 10 minutes to fix it. Wherein, the amount of mixed DNA was 60 ul of the QPCR product. A 20 mM NaOH solution was added thereto to make it into single strand DNA.

And, DNA including modified nucleic acid was synthesized by the same method as Example 1.1 Synthesis of Library, and used for the next round. Total 8 SELEX rounds were conducted, and for more selective binding, at 4^{th} to 6^{th} and 7^{th} to 8^{th} rounds, DNA-protein (integrin αᵥβ₃) complex was diluted in a 10 mM DxSO₄ (sigma) solution to 1/200, 1/400 respectively, to select DNA aptamer.

(5) Pool Binding Assay:

In order to examine binding capacity of DNA pool that has passed SELEX rounds with integrin αᵥβ₃, filter binding assay was conducted. The pools of 6 round and 8 round were labeled with α-P³²ATP (Perkin Elmer) and TdT (Terminal deoxynucleotidyl transferase, NEB) at the end. 1 μM of the library DNA obtained through the SELEX process, 0.25 μL, α-P³²ATP (5 μM, perkinelmer), 0.25 μL TdT, and 10×NEB buffer4 (NEB) 10 μL reaction volume were reacted at 37° C. for 30 minutes, and incubated at 70° C. for 10 minutes to inactivate TdT. The labeled DNA pool was purified using Micro spin G-50 column (GE healthcare).

20,000 cpm of the labeled DNA pool were put in 100 μL 1×SB buffer (200 mM HEPES, 510 mM NaCl, 25 mM KCl, 25 mM MgCl₂), and slowly cooled from 95° C. to 37° C. at a rate of 0.1° C./sec. And, integrin αᵥβ₃ (R&D systems, 3050-AV) was serially diluted to 12 points at 100 nM using buffer (200 mM HEPES, 510 mM NaCl, 25 mM KCl, 25 mM MgCl₂), and then, 30 μL of the above heated and cooled DNA pool was respectively added and reacted at 37° C. for 30 minutes. A nylon membrane (GE healthcare) was spotted with 2 μL of the complex of DNA and integrin αᵥβ₃, and then, 5.5 μL of zorbax resin (Agilent) was added thereto. And, it was put in a Durapore filter (Millipore) previously wetted with 50 μL of 1×SB buffer (200 mM HEPES, 510 mM NaCl, 25 mM KCl, 25 mM MgCl₂) and vacuum was applied. And, the membrane filter was washed with 100 μL of 1× selection buffer (200 mM HEPES, 510 mM NaCl, 25 mM KCl, 25 mM MgCl₂). The filter plate was exposed to an image plate overnight, and then, images were quantified with FLA-5100 (Fuji).

Binding affinity between integrin αᵥβ₃ and DNA pool that has passed SELEX rounds was shown in the following Table 1. The binding affinity was calculated using SigmaPlot 11 (Systat Software Inc.) from the values obtained through the filter binding assay, and in the Table 1, Bₘₐₓ denotes the ratio of bound aptamer compared to input, and K_{d} (dissociation constant) denotes affinity.

TABLE 1

| | Library | pool binding |
|---|---|---|
| Bₘₐₓ | 3.23E−11 | 0.45 |
| K_{d} (nM) | 6500.86 | 43.3 |

In the Table, the library is DNA having a random base sequence with benzyl group-modified nucleic acid, and the pool binding means ssDNA Pool obtained after 8 Round among the previously described SELEX steps using a specific library.

(6) Analysis of Base Sequence of Integrin αᵥβ₃ Aptamer:

The 8 Round ssDNA Pool having the highest binding affinity after passing 8 SELEX rounds was amplified with double strand DNA by the above mentioned QPCR method, and then, cloned using a TA cloning kit (SolGent). And, it was sequenced with a M13 primer (CAGGAAACAGCTATGAC; SEQ ID NO: 67) existing on the vector to obtain the following sequence.

The obtained DNA aptamer very specifically binding to integrin αᵥβ₃ has a base sequence of

5′-TCAGCCGCCAGCCAGTTC-[Core sequence]-GACCAGAGCACCACAGAG-3′ (SEQ ID NO: 61),

wherein the core sequence is as described in the following Table 2, and among them, 5 denotes benzyl-dU.

TABLE 2

| | Description | |
|---|---|---|
| # | (Clone No.) | Core sequence |
| #1 | S003-A4- | 5CGGAGGC555ACA5CGG5AACCGAGAC |
| | 001-T7_A01 | 55AGGAC5G55G (SEQ ID NO: 1) |
| #2 | S003-A4- | 5C5CA55C55ACACAAGGCCAGA5AAAG |
| | 002-T7_B01 | 5G5AGCAAAG55 (SEQ ID NO: 2) |
| #3 | S003-A4- | 5AG55G5ACA55C5GAG555AGAGCAAA |
| | 003-T7_C01 | 5AA5AGAG5CCA (SEQ ID NO: 3) |
| #4 | S003-A4- | 5AC5AAACACGCAGAC5GAAA5555ACA |
| | 004-T7_D01 | 5CGG5AACAG5C (SEQ ID NO: 4) |
| #5 | S003-A4- | 5555AA5C55C5C5G5CAGA5GGC5GG5 |
| | 005-T7_E01 | AGGG5G5A5GAC (SEQ ID NO: 5) |
| #6 | S003-A4- | CGAGGGAG55A5GGAGA55G5G55G555 |
| | 006-T7_F01 | AAGG5CGGAAC5 (SEQ ID NO: 6) |
| #7 | S003-A4- | CGAGGGAG55A5GGGGA55G5G55G555 |
| | 007-T7_G01 | AAGG5CGGAAC5 (SEQ ID NO: 7) |
| #8 | S003-A4- | 5555AA5C55C5C5G5CAGA5GGC5GG5 |
| | 008-T7_H01 | AGGG5G5A5GAC (SEQ ID NO: 8) |
| #9 | S003-A4- | AG55GCCAA555GCAGCC5AGGA5ACG5 |
| | 009-T7_A02 | 555CGAAAC5GCA (SEQ ID NO: 9) |
| #10 | S003-A4- | CCG5CAGCGCGG55CGAAGG5ACAA555 |
| | 010-T7_B02 | 5AGA5CGC5AAG (SEQ ID NO: 10) |
| #11 | S003-A4- | 5555AA5C55C5C5G5CAGA5GGC5GG5 |
| | 011-T7C02 | AGGG5G5A5GAC (SEQ ID NO: 11) |
| #12 | S003-A4- | AG55GCCAA555GCAGCC5AGGA5ACG5 |
| | 012-T7_D02 | 555CGAAAC5GCA (SEQ ID NO: 12) |
| #13 | S003-A4- | 5555AA5C55C5C5G5CAGA5GGC5GG5 |
| | 013-T7_E02 | AGGG5G5A5GAC (SEQ ID NO: 13) |
| #14 | S003-A4- | 5555AA5C55C5C5G5CAGA5GGC5GG5 |
| | 014-T7_F02 | AGGG5G5A55AC (SEQ ID NO: 14) |
| #15 | S003-A4- | ACG5AAAGGAGACGGA5555GACCCG5G |
| | 015-T7_G02 | 5A5AC5CGACGC (SEQ ID NO: 15) |
| #16 | S003-A4- | GA555CGGAA5AAGGCC55A5GAACCA5 |
| | 016-T7_H02 | GAGCC5G5C5C (SEQ ID NO: 16) |
| #17 | S003-A4- | 5G5GA5A5G5C55G55AAGC55C5GA5G |
| | 017-T7_A03 | A5GCAGGGC5GG (SEQ ID NO: 17) |
| #18 | S003-A4- | 5C5CC55C55ACCCCG5G5AGCAAAGA5 |
| | 018-T7_B03 | 5CAGC5GAGGAG (SEQ ID NO: 18) |
| #19 | S003-A4- | 5AA5AAGCCAC5CGGCG5CAC5G5AG5A |
| | 019-T7_C03 | 5G555A5C5A5C (SEQ ID NO: 19) |
| #20 | S003-A4- | AGCG5GAGACAGG5G5GAGGAGGCAA55 |
| | 020-T7_D03 | 55ACA5AGG5AA (SEQ ID NO: 20) |
| #21 | S003-A4- | CGAGGGAG55A5GGAGA55G5G55G555 |
| | 021-T7_E03 | AAGG5CGGAAC5 (SEQ ID NO: 21) |
| #22 | S003-A4- | AG55GCCAA555GCAGCC5AGGA5ACG5 |
| | 022-T7_F03 | 555CGAAAC5GCA (SEQ ID NO: 22) |
| #23 | S003-A4- | AG55GCCAA555GCAGCC5AGGA5ACG5 |
| | 023-T7_G03 | 555CGAAAC5GCA (SEQ ID NO: 23) |
| #24 | S003-A4- | CGAGGGAG55A5GGAGA55G5G55G555 |
| | 024-T7_H03 | AAGG5CGGAAC5 (SEQ ID NO: 24) |
| #25 | S003-A4- | G5555AAGAAA55AGCACACCG55GAC5 |
| | 025-T7_A04 | 5G555AG5GGCG (SEQ ID NO: 25) |
| #26 | S003-A4- | G5555AAGAAA55AGCACA5CG55GAC5 |
| | 026-T7_B04 | 5G555AG5GGCG (SEQ ID NO: 26) |
| #27 | S003-A4- | AGG5CAC5A5GA5555GACCCG5G555G |
| | 027-T7_C04 | C5CGACGCG5AA (SEQ ID NO: 27) |
| #28 | S003-A4- | AGG5CAC5A5GA5555GACCCG5G555A |
| | 028-T7_D04 | C5CGACGCGCAA (SEQ ID NO: 28) |
| #29 | S003-A4- | 5G5GA5A5G5C55G55AAGC55C5GA5G |
| | 029-T7_E04 | A5ACCGGGC5GG (SEQ ID NO: 29) |
| #30 | S003-A4- | G5555AAGAAA55AGCACACCG55GAC5 |
| | 030-T7_F04 | 5G555AG5GGCG (SEQ ID NO: 30) |
| #31 | S003-A4- | 5555AA5C55C5C5G5CAGA5GGC5GG5 |
| | 031-T7_G04 | AGGG5G5A5GAA (SEQ ID NO: 31) |
| #32 | S003-A4- | 55CAGACCAA55A5GG5AA555C5CAAA |
| | 032-T7_H04 | 5C5GAG5G5CA5 (SEQ ID NO: 32) |
| #33 | S003-A4- | 5CG5A5555GACCCG5G5A5CC5CGA5G |
| | 033-T7_A05 | CGG55AGCAGCA (SEQ ID NO: 33) |
| #34 | S003-A4- | 5AA5AAGCCAC5CGGCG5CAC5G5AG5A |
| | 034-T7_B05 | 5G555A5C5A5C (SEQ ID NO: 34) |
| #35 | S003-A4- | G5555AAGAAA55AGCACACCG55GAC5 |
| | 035-T7_C05 | 5G555AG5GGCG (SEQ ID NO: 35) |
| #36 | S003-A4- | G5555AAGAAA55AGCACACCG55GAC5 |
| | 036-T7_D05 | 5G555AG5GGCG (SEQ ID NO: 36) |
| #37 | S003-A4- | CGAGGGAG55A5G5AGA55G5G55G555 |
| | 037-T7_E05 | AAGG5C5GAAC5 (SEQ ID NO: 37) |
| #38 | S003-A4- | 5C5CA55C55ACACAAGGCCAGAGAAAG |
| | 038-T7_F05 | 5G5AGCAAAG55 (SEQ ID NO: 38) |
| #39 | S003-A4- | GAC5555ACA5CGG5AAAGAAC5CAGA5 |
| | 039-T7_G05 | A5GCACAAG55A (SEQ ID NO: 39) |
| #40 | S003-A4- | 5CGAGCCA5GG5CGAGCCCCA5555ACA |
| | 040-T7_H05 | 5CGG5AAGGGC5 (SEQ ID NO: 40) |
| #41 | S003-A4- | G5555AAGAAA55AGCACACCG55GAC5 |
| | 041-T7_A06 | 5G555AG5GGCG (SEQ ID NO: 41) |
| #42 | S003-A4- | 5G5GA5A5G5C55G55AAGC55C5GA5G |
| | 042-T7_B06 | A5GCAGGGC5GG (SEQ ID NO: 42) |
| #43 | S003-A4- | CGAGGGAG55A5GGAGA55G5G55G555 |
| | 043-T7_C06 | AAGG5CGGAAC5 (SEQ ID NO: 43) |
| #44 | S003-A4- | AGC5AACGACA5555ACA5CGG5AAG5C |
| | 044-T7_D06 | AAACC5CAGCAC5 (SEQ ID NO: 44) |
| #45 | S003-A4- | 5AA5C5GG5AG555AAGCACA55G5GA5 |
| | 045-T7_E06 | 5GCACGCGGA5G555GA5 |
| | | (SEQ ID NO: 45) |
| #46 | S003-A4- | GA555CGGAA5AAGGCC55A5GAACCA |
| | 046-T7_F06 | 5GAGCC5G5C5C (SEQ ID NO: 46) |
| #47 | S003-A4- | AG55GCCAA555GCAGCC5AGGA5ACG5 |
| | 047-T7_G06 | 555CGAAAC5GCA (SEQ ID NO: 47) |
| #48 | S003-A4- | 5C5GGCAAC5555ACA5CGG5AAGCC5A |
| | 048-T7_H06 | 55GAGCGCGAC5 (SEQ ID NO: 48) |
| #49 | S003-A4- | 5555AA5C55C5C5G5CAGA5GGC5GG5 |
| | 049-T7_A07 | AGGG5G5G5GAC (SEQ ID NO: 49) |
| #50 | S003-A4- | CGAGGGAG55A5GGAGA55G5G55G555 |
| | 050-T7_B07 | AAGG5CGGAAC5 (SEQ ID NO: 50) |
| #51 | S003-A4- | 5A55GGGAGG5GGGGGCCA555ACA5AG |
| | 051-T7_C07 | G5AACAGCCAC5 (SEQ ID NO: 51) |
| #52 | S003-A4- | 5555AA5C55C5C5G5CAGA5GGC5GG5 |
| | 052-T7_D07 | AGGG5G5A5GAC (SEQ ID NO: 52) |
| #53 | S003-A4- | AG55G5CAA555GCAGCC5A5GA5ACG5 |
| | 053-T7_E07 | 555CGAAAC5GCA (SEQ ID NO: 53) |
| #54 | S003-A4- | CGAACGGAA5GGA5CAG5CC5GGGCAA5 |
| | 054-T7_F07 | 555ACA5AGG5AA (SEQ ID NO: 54) |
| #55 | S003-A4- | CGAGGGAG55A5GGAGA55G5G55G555 |
| | 055-T7_G07 | AAGG5CGGAAC5 (SEQ ID NO: 55) |
| #56 | S003-A4- | AGGC5AGCGGGACAG5A555GAACCG5G |
| | 056-T7_H07 | 5A5CC5CGACGC (SEQ ID NO: 56) |

5 = Benzyl-dU(BzdU):[5-(N-Benzylaminocarbonylamide)-2′-deoxyuridine] 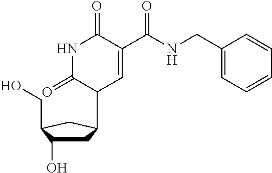
A = 2′-deoxyAdenosine
G = 2′-deoxyGuanosine
C = 2′-deoxyCytidine
T = 2′-deoxyThymidine(Thymidine)

The discovered integrin αᵥβ₃ aptamers were classified as analogous family (sequence homology is based on 85% homology of base sequence):

TABLE 3
69.64.00% (39/56) Multi-Copy

| | | Num | | |
|---|---|---|---|---|
| # | Description | ber | (%) | core seq. |
| [11] | S003-A4-011-T7_C02 | 8 | 14.29 | 5555AA5C55C5C5G5CAGA5GGC5GG5A |
| | | | | GGG5G5A5GAC |
| | | | | (SEQ ID NO: 11) |
| | | | | [5, 8, 11, 13, 14, 31, 49, 52] |
| [12] | S003-A4-012-T7_D02 | 6 | 10.72 | AG55GCCAA555GCAGCC5AGGA5ACG |
| | | | | 5555CGAAAC5GCA |
| | | | | (SEQ ID NO: 12) |
| | | | | [9, 12, 22, 23, 47, 53] |
| [16] | S003-A4-016-T7_H02 | 2 | 3.57 | GA555CGGAA5AAGGCC55A5GAACCA |
| | | | | 5GAGCC5G5C5C |
| | | | | (SEQ ID NO: 16) [16, 46] |
| [17] | S003-A4-017-T7_A03 | 3 | 5.36 | 5G5GA5A5G5C55G55AAGC55C5GA5GA |
| | | | | 5GCAGGGC5GG |
| | | | | (SEQ ID NO: 17) [17, 29, 42] |
| [19] | S003-A4-019-T7_C03 | 2 | 3.57 | 5AA5AAGCCAC5CGGCG5CAC5G5AG5 |
| | | | | A5G555A5C5A5C |
| | | | | (SEQ ID NO: 19) [19, 34] |
| [2] | S003-A4-002-T7_B01 | 2 | 3.57 | 5C5CA55C55ACACAAGGCCAGA5AAA |
| | | | | G5G5AGCAAAG55 |
| | | | | (SEQ ID NO: 2) [2, 38] |
| [21] | S003-A4-021-T7_E03 | 8 | 14.29 | CGAGGGAG55A5GGAGA55G5G55G555 |
| | | | | AAGG5CGGAAC5 |
| | | | | (SEQ ID NO: 21) |
| | | | | [6, 7, 21, 24, 37, 43, 50, 55] |
| [25] | S003-A4-025-T7_A04 | 6 | 10.71 | G5555AAGAAA55AGCACACCG55GAC5 |
| | | | | 5G555AG5GGCG |
| | | | | (SEQ ID NO: 25) |
| | | | | [25, 26, 30, 35, 36, 41] |
| [27] | S003-A4-027-T7_C04 | 2 | 3.57 | AGG5CAC5A5GA5555GACCCG5G555G |
| | | | | C5CGACGCG5AA |
| | | | | (SEQ ID NO: 27) [27, 28] |

In the case of #11 clone, among 56 sequences, identical base sequence repeatedly appeared 8 times. And, for #21, repeated base sequence appeared 8 times, for #12, #25, 6 times, for #17, 3 times, and for #2, #16, #19, #27, 2 times.

Sequence similarity of the discovered aptamers was confirmed by measuring repeatedly occurring parts among the base sequences of the clones and the frequency, and the results are as follows:

| 57.144% (32/56) Families | |
|---|---|
| [44]S003-A4-044-T7_D06 Count: 1 0.02% | |
| AGCTAACGACATTTTACATCGGTAAGTCAAACCTCAGCACT | |
| ***************** | |
| TTTACATCGGTAA | Pattern_6 X 6 times |
| ATTTTACAT | Pattern_7 X 5 times |
| ACATAGGTAA | Pattern_8 X 9 times |
| TTTTACATCGGTAA | Pattern_13 X 5 times |
| ACTTTTACATCGGTAA | Pattern_14 X 5 times |
| CATTTTACATCGGTAAG | Pattern_15X3 times |
| Score: 33 | |
| [40]S003-A4-040-T7_1105 Count: 1 0.02% | |
| TCGAGCCATGGTCGAGCCCCATTTTACATCGGTAAGGGCT | |
| ***************** | |
| TTTACATCGGTAA | Pattern_6 X 6 times |
| ATTTTACAT | Pattern_7 X 5 times |
| ACATAGGTAA | Pattern_8 X 9 times |
| TTTTACATCGGTAA | Pattern_13 X 5 times |
| ACTTTTACATCGGTAA | Pattern_14 X 5 times |
| CATTTTACATCGGTAAG | Pattern_15 X 3 times |
| Score: 33 | |
| [4]S003-A4-004-T7_D01 Count: 1 0.02% | |
| TACTAAACACGCAGACTGAAATTTTACATCGGTAACAGTC | |
| ******************* | |
| TTTACATCGGTAA | Pattern_6 X 6 times |
| ATTTTACAT | Pattern_7 X 5 times |
| ACATAGGTAA | Pattern_8 X 9 times |
| TTTTACATCGGTAA | Pattern_13 X 5 times |
| ACTTTTACATCGGTAA | Pattern_14 X 5 times |
| GGTAACAG | Pattern_16 X 3 times |
| Score: 33 | |
| [48]S003-A4-048-T7_H06 Count: 1 0.02% | |
| TCTGGCAACTTTTACATCGGTAAGCCTATTGAGCGCGACT | |
| ***************** | |
| TTTACATCGGTAA | Pattern_6 X 6 times |
| ACATAGGTAA | Pattern_8 X 9 times |
| TTTTACATCGGTAA | Pattern_13 X 5 times |
| ACTTTTACATCGGTAA | Pattern_14 X 5 times |
| CATTTTACATCGGTAAG | Pattern_15 X 3 times |
| Score: 28 | |
| [39]S003-A4-039-T7_G05 Count: 1 0.02% | |
| GACTTTTACATCGGTAAAGAACTCAGATATGCACAAGTTA | |
| **************** | |
| TTTACATCGGTAA | Pattern_6 X 6 times |
| ACATAGGTAA | Pattern_8 X 9 times |
| TTTTACATCGGTAA | Pattern_13 X 5 times |
| ACTTTTACATCGGTAA | Pattern_14 X 5 times |
| Score: 25 | |
| [54]S003-A4-054-T7_F07 Count: 1 0.02% | |
| CGAACGGAATGGATCAGTCCTGGGCAATTTTACATAGGTAA | |
| ******************* | |
| CAATTTTA | Pattern_1 X 3 times |
| ATTTTACAT | Pattern_7 X 5 times |
| ACATAGGTAA | Pattern_8 X 9 times |
| GGCAATTTTACATAGGTAA | Pattern_9X2 times |
| Score: 19 | |
| [20]S003-A4-020-T7_D03 Count: 1 0.02% | |
| AGCGTGAGACAGGTGTGAGGAGGCAATTTTACATAGGTAA | |
| ******************* | |
| CAATTTTA | Pattern_l X 3 times |
| ATTTTACAT | Pattern_7 X 5 times |
| ACATAGGTAA | Pattern_8 X 9 times |
| GGCAATTTTACATAGGTAA | Pattern_9X 2 times |
| Score: 19 | |
| [1]S003-A4-001-T7_A01 Count: 1 0.02% | |
| TCGGAGGCTTTACATCGGTAACCGAGACTTAGGACTGTTG | |
| **************** | |
| TTTACATCGGTAA | Pattern_6 X 6 times |
| ACATAGGTAA | Pattern_8 X 9 times |
| GGTAACAG | Pattern_16 X 3 times |
| Score: 18 | |
| [15]S003-A4-015-T7_G02 Count: 1 0.02% | |
| ACGTAAAGGAGACGGATTTTGACCCGTGTATACTCGACGC | |
| ************************** | |
| CCCGTGTA | Pattern_2 X 3 times |
| GATTTTGACCCGTGT | Pattern_3 X 3 times |
| ATTTTGACCCGTGTAT | Pattern_4 X 2 times |
| CTCGACGC | Pattern_11 X 4 times |
| CCGTGTATCCTCGA | Pattern_12 X 3 times |
| Score: 15 | |
| [25]S003-A4-025-T7_A04 Count: 60.11% | |
| GTTTTAAGAAATTAGCACACCGTTGACTTGTTTAGTGGCG | |
| ******* | |
| TTGTTTA | Pattern_10 X 14 times |
| Score: 14 | |
| [21]S003-A4-021-T7_E03 Count: 80.14% | |
| CGAGGGAGTTATGGAGATTGTGTTGTTTAAGGTCGGAACT | |
| ******* | |
| TTGTTTA | Pattern_10 X 14 times |
| Score: 14 | |
| [51]S003-A4-051-T7_C07 Count: 1 0.02% | |
| TATTGGGAGGTGGGGGCCATTTACATAGGTAACAGCCACT | |
| ************* | |
| ACATAGGTAA | Pattern_8 X 9 times |
| GGTAACAG | Pattern_16 X 3 times |
| Score: 12 | |
| [33]S003-A4-033-T7_A05 Count: 1 0.02% | |
| TCGTATTTTGACCCGTGTATCCTCGATGCGGTTAGCAGCA | |
| ********************** | |
| CCCGTGTA | Pattern_2 X 3 times |
| ATTTTGACCCGTGTAT | Pattern_4 X 2 times |
| CCGTGTATCCTCGA | Pattern_12 X 3 times |
| Score: 8 | |
| [56]S003-A4-056-T7_H07 Count: 1 0.02% | |
| AGGCTAGCGGGACAGTATTTGAACCGTGTATCCTCGACGC | |
| ***************** | |
| CTCGACGC | Pattern_11 X 4 times |
| CCGTGTATCCTCGA | Pattern_12 X 3 times |
| Score: 7 | |
| [27]S003-A4-027-T7_C04 Count: 20.04% | |
| AGGTCACTATGATTTTGACCCGTGTTTGCTCGACGCGTAA | |
| *************** ******** | |
| GATTTTGACCCGTGT | Pattern_3 X 3 times |
| CTCGACGC | Pattern_11 X 4 times |
| Score: 7 | |
| [18]S003-A4-018-T7_B03 Count: 1 0.02% | |
| TCTCCTTCTTACCCCGTGTAGCAAAGATTCAGCTGAGGAG | |
| *************** | |
| CCCGTGTA | Pattern_2 X 3 times |
| TTCTTAC | Pattern_5 X 3 times |
| Score: 6 | |
| [10]S003-A4-010-T7_B02 Count: 1 0.02% | |
| CCGTCAGCGCGGTTCGAAGGTACAATTTTAGATCGCTAAG | |
| ******** | |
| CAATTTTA | Pattern _1X 3 times |
| Score: 3 | |
| [2]S003-A4-002-T7_B01 Count: 20.04% | |
| TCTCATTCTTACACAAGGCCAGATAAAGTGTAGCAAAGTT | |
| ******* | |
| TTCTTAC | Pattern_5 X 3 times |
| Score: 3 | |

5.36% (3/56) Orphans
[3]S003-A4-003-T7_C01 1 0.02%
TAGTTGTACATTCTGAGTTTAGAGCAAATAATAGAGTCCA
[32]S003-A4-032-T7_H04 1 0.02%
TTCAGACCAATTATGGTAATTTCTCAAATCTGAGTGTCAT
[45]S003-A4-045-T7_E06 1 0.02%
TAATCTGGTAGTTTAAGCACATTGTGATTGCACGCGGATGTTTGAT

(7) Clone Binding Assay:

In order to examine binding affinity of clone having repeatedly observed base sequence, filter binding assay was conducted by the same method as pool binding assay. The binding affinity was calculated using SigmaPlot 11 (Systat Software Inc.) from the value obtained through the filter binding assay, and the results are shown in the following Table 4. In the Table 4, Bₘₐₓ denotes the amount of binding aptamer compared to input, wherein as the value is closer to 1, good performance is indicated, and K_{d} (dissociation constant) denotes affinity, wherein the lower value indicates the higher binding capacity.

TABLE 4

| | #11 | #12 | #21 | #25 | 8R Pool | Bz library |
|---|---|---|---|---|---|---|
| Bₘₐₓ | 0.56 | 0.69 | 0.88 | 0.82 | 0.45 | 3.23E−11 |
| K_{d} (nM) | 38.02 | 23.22 | 58.95 | 17.57 | 43.3 | 6500.86 |

Assay was conducted with total 5 kinds of clones, one of them could not obtain K_{d}, and #25 clone (S003-A4-025-T7_A04) exhibited K_{d} of 17.57 nM, the highest binding capacity to target protein.

(8) Determination of Optimum Aptamer Sequence Through Truncation from Full Length Aptamer:

DNA aptamer cloned through the SELEX process has a sequence length of about 80mer, and such a length was selected as having a suitable range of dissociation constant (K_{d}) with target protein. Among them, the best evaluated clone #25 Clone (2100-25-)) was used to synthesize 50mer aptamer 2100-25-02 having a base sequence of

5′-AGTTC-[Core sequence]-GACCA-3′(SEQ ID NO: 62)

partially including a primer region (See Table 5).

In Table 5, 5 in the base sequence denotes benzyl-dU.

TABLE 5

| | | | Calculated | Observed |
|---|---|---|---|---|
| | | | molecular | molecular |
| | | | weight | weight |
| Sequence ID | Sequence | Sample name | (Da) | (Da) |
| HO-2100-25-2 | AGTTCG5555AAG | Intigrin_{aVb3} | 17097.65 | 17099.53 |
| | AAA55AGCACACC | 50 mer | | |
| | G55GAC55G555AG | | | |
| | 5GGCGGACCA | | | |
| | (SEQ ID NO: 63) | | | |

(9) Synthesis of Integrin αᵥβ₃ Aptamer:

Aptamer was self synthesized by a Solid Phase Oligo Synthesis method using a Mermade 12 synthesizer (Bioautomation Corp.), which is a solid phase synthesizer only for nucleic acid.

(10) Synthesis and Separation/Purification/Identification of Integrin αᵥβ₃ Aptamer and QC:

The above discovered aptamers having modified nucleic acid were synthesized using an oligonucleotide synthesizer (Bioautomation, Mermade12) by solide phase-cyanoethyl phosphoramidite chemistry. Thereafter, CPG (200 nmole synthesis column, 1000 A (MM1-1000-)) was put in a cleavage solution [t-butylamine:methanol:water (1:1:2 volume ratio)], cleavage/deprotection was conducted at 70° C. for 5 hours, followed by vacuum drying, and then, separation/purification using HPLC (GE, AKTA basic). The column used was RP-C18 column (Waters, Xbridge OST C18 10×50 mm), and 0.1M TEAB/Acetonitrile Buffer was used under conditions of UV 254 nm/290 nm, flow rate: 5 ml/min, temperature: 65° C. The exact molecular weights of the aptamers were measured with LC-ESI MS spectrometer (Waters HPLC systems(Waters)+Qtrap2000(ABI)) within the error range of 0.02%, and according to purity determination using HPLC, 80-90% could be obtained.

### Example 2

### Preparation of Integrin αᵥβ₃ Aptamer Conjugated Magnetic Nanoparticles

2.1: Preparation of High Sensitivity Magnetic Nanocrystals Using Thermal Decomposition

7 nm magnetic nano-crystals (MNCs) were synthesized by heating each 0.6 moles of dodecylic acid and dodecyl amine in a benzylether solvent of 215° C. with iron triacetyl acetonate and manganese triacetyl acetonate (Aldrich) for 2 hours, followed by thermal decomposition at 315° C. for 1 hour.

The benzylether solution comprising 7 nm MNCs (10 mg/ml) and dodecylic acid (0.2 moles), dodecyl amine (0.1 mole), iron triacetyl acetonate and manganese triacetyl acetonate was heated at 115° C. for 30 minutes, at 215° C. for 2 hours, and at 315° C. for 1 hour to prepare 12 nm MNCs.

2.2: Polymerization of Amphiphilic Compound Having a Functional Group Capable of Binding with Integrin αᵥβ₃ Aptamer Bonded to the Hydrophilic Region

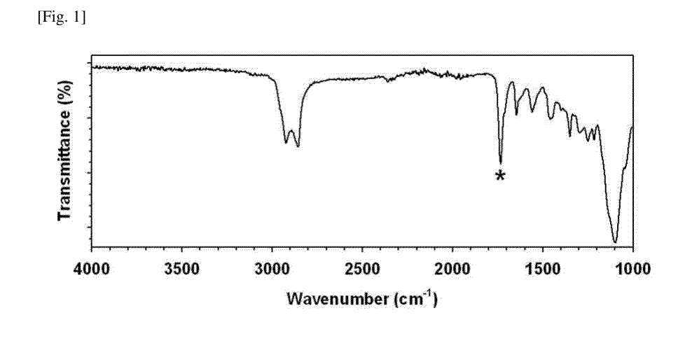

5 g of polyoxyethylene sorbitan monooleate (Polysorbate 80), 1.5 g of succinic anhydride (SA), 1.8 g of 4-dimetlaminopridine (DMAP), and 1.5 g of triethylamine (TEA) were added to 120 mL of a 1,4-dioxane solvent, and the mixture was stirred using a magnetic bar for 48 hours. After the reaction was completed, 1,4-dioxane was removed by lyophilization, and the solvent-free product was dispersed in carbon tetrachloride (CCl₄) and then filtered to remove remaining reactants. The filtered solution was precipitated through ethyl ether, and the precipitate was dried to obtain a compound (tri-carboxylated polysorbate 80, P80-triCOOH) that has a functional group (COOH) to which integrin αᵥβ₃ aptamer (Aptamer_{αvβ3}, Apt_{αvβ3}) can bind and simultaneously exhibits amphiphilicity. The structure of ester (—COO—) that was formed when preparing P80-triCOOH was confirmed through infrared spectrum, and the result is shown in FIG. 1.

2.3: Preparation of Magnetic Nanoparticles Coated with an Amphiphilic Compound Capable of Binding with Integrin αᵥβ₃ Aptamer

10 mg of MNCs prepared in Example 2.1, dissolved in an oil phase of 10 mL n-hexane, and 100 mg of P80-triCOOH prepared in Example 2.2, dissolved in an aqueous phase were mixed, and the mixture was saturated by 450 W ultrasonic waver for 10 minutes. The emulsion was stirred at room temperature for 12 hours to evaporate the oil phase, and centrifugation using a centrifugal filter (Centriprep YM-3, 3 kDa NMWL) (RPM: 18,000) afforded excessive P80-triCOOHH-removed magnetic nanoparticles (MNPs) coated with an amphiphilic compound capable of binding with aptamer.

2.4: Preparation of Magnetic Nanoparticles Conjugated with Integrin αᵥβ₃ Aptamer Capable of Diagnosing Cancer and Cancer Metastasis

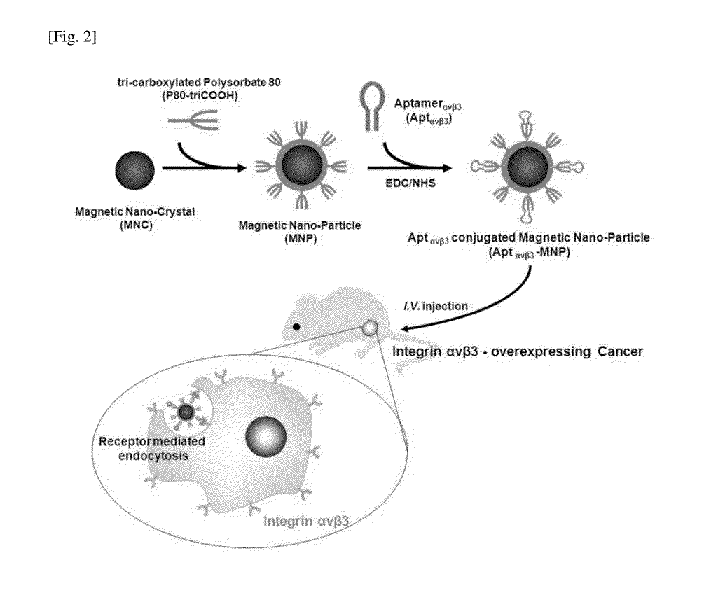

The process of conjugating integrin αᵥβ₃ aptamer (Aptamer_{αvβ3}, Apt_{αvβ3}) to the functional group (COOH) on the surface of NMPs prepared in Example 2.3 to prepare Aptamer_{αvβ3}-conjugated magnetic nanoparticles (Apt_{αvβ3}-MNPs) is shown in FIG. 2. In 2.5 mL of a phosphate buffer solution (10 mM, pH7.4), MNPs prepared in Example 2.3, 38.2 μmol of 1-ethyl-3-(3-dimethylaminopropyl)-carbodiimide (EDC), 38.2 μmol of sulfo-n-hydroxysuccinimide (sulfo-NHS), and 0.2 mg (11.5 nmol) of Apt_{αvβ3} were dissolved, and then, reacted at 4° C. for 4 hours. After 4 hours, non-bound Apt_{αvβ3} was separated from the mixture using a Centrifugal filter (Amicon Ultra).

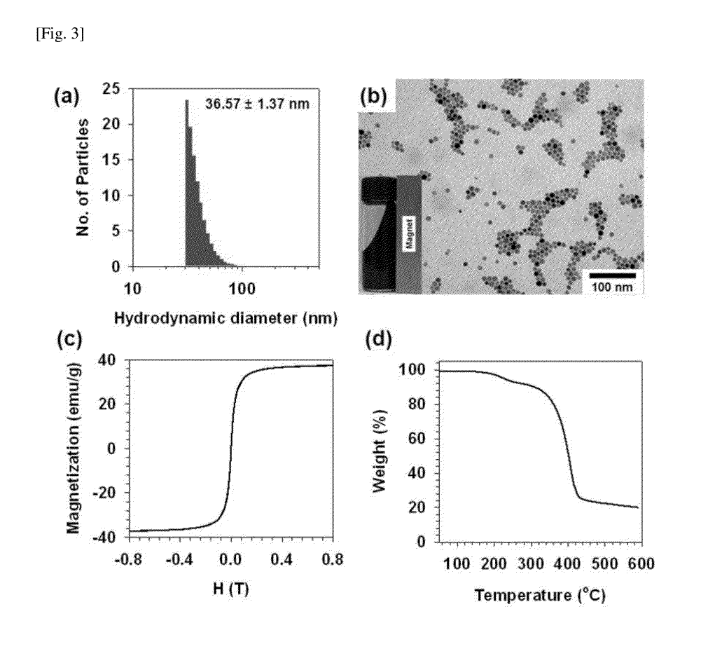

The size of Apt_{αvβ3}-MNPs was measured by dynamic laser light scattering, the shape of the particles was confirmed through transmission electron microscope, and super paramagnetism was confirmed using a vibration sample magnetometer (VSM), and then, the content of magnetic crystals was measured by a thermogravimetric analyzer (TGA), and shown in FIGS. 3a, 3b, 3c, and 3d.

### Example 3

### Cancer Cell Imaging in an Animal Model Using Aptamer_{αvβ3}-Conjugated Magnetic Nanoparticles

3.1: Analysis of MR Contrast Effect of Aptamer_{αvβ3}-Conjugated Magnetic Nanoparticles

In order to confirm the applicability of the prepared Apt_{αvβ3}-MNPs as an MRI contrast agent, the MR contrast effect of the magnetic nano complex was examined through measurement of r2 (T2 relativity coefficients). Specifically, an MR image test was conducted using 1.5 T clinical MRI instrument (Intera, Philips Medical System) having Micro-47 surface coil. The r2 value (unit of mM⁻¹s⁻¹) of the magnetic nano complex was measured at room temperature using CPMG (Carr-Purcell-Meiboom-Gill) sequence (TR=10 s, 32 echoes with 12 ms even echo space, number of acquisitions=1, point resolution of 156×156 μm, section thickness of 0.6 mm).

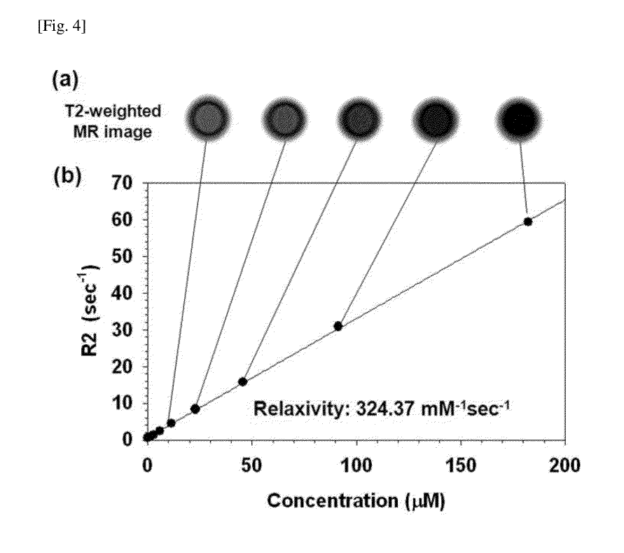

As shown in FIG. 4a, at 1.5T, as the concentration of Apt_{αvβ3}-MNPs increase, remarkably dark MR contrast was provided, and FIG. 4b shows that as the concentration increases, r2 clearly increases. Thus, it was confirmed that Apt_{αvβ3}-MNPs as an MR imaging probe has sufficient magnetic property for molecular imaging.

3.2: Evaluation of Stability of Aptamer_{αvβ3}-Conjugated Magnetic Nanoparticles

For stability evaluation of the prepared Apt_{αvβ3}-MNPs, they were dispersed under serum containing conditions at various concentrations, and then, the particle size was measured. MNPs conjugated with well known peptide, cyclo (Arg-Gly-Asp-D-Phe-Lys) (cRGD), (cRGD-MNPs) was simultaneously tested for cell viability.

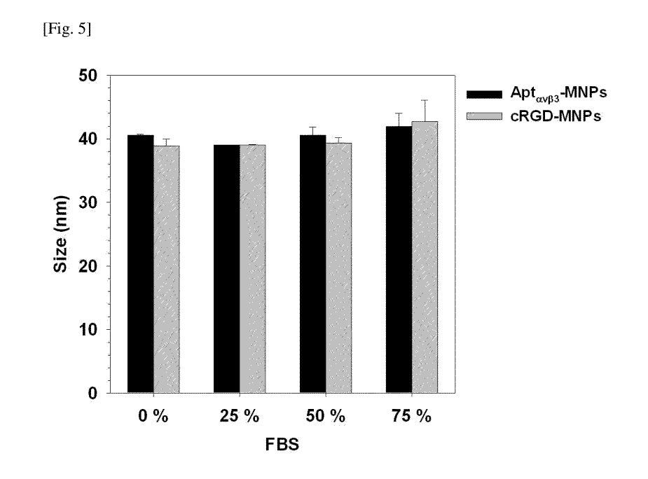

As shown in FIG. 5, particle size did not change even if concentration conditions changed, thus confirming that the particles may have stability even under intracellular conditions.

3.3: Confirmation of Cell Viability of Aptamer_{αvβ3}-Conjugated Magnetic Nanoparticles

The cell viability of the Apt_{αvβ3}-MNPs prepared in Example 3.1 was tested, and for comparison of stability, the cell viability of MNPs conjugated with cyclo (Arg-Gly-Asp-D-Phe-Lys) (cRGD) (cRGD-MNPs), of which integrin αᵥβ₃ targetability is well known, was simultaneously tested. In 96-wells, 1×10⁴ cells (PAE/KDR) were seeded per well, and cultured in MEM culture medium containing 5% fetal bovine serum (FBS) and 1% antibiotics at 37° C., 5% CO₂ conditions. Thereafter, the cells were treated with various concentrations of Apt_{αvβ3}-MNPs and cRGD-MNPs, and then, additionally cultured for 24 hours.

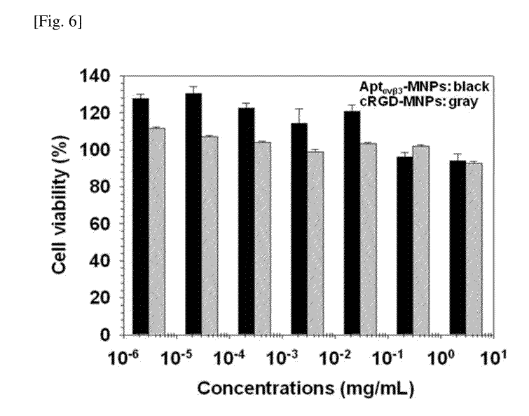

For cytotoxicity of Apt_{αvβ3}-MNPs and cRGD-MNPs, the degree of cell growth inhibition was measured by 3-(4,5-dimethylthiazol-2-yl)-2,5-diphenyltetrazolium bromide (MTT) assay. As shown in FIG. 6, it was confirmed that neither Apt_{αvβ3}-MNPs and cRGD-MNPs exhibited cytotoxicity even at high concentrations.

3.4: Confirmation of Targetability of Aptamer_{αvβ3}-Conjugated Magnetic Nanoparticles

The targetability of the Apt_{αvβ3}-MNPs prepared in Example 2.4 to integrin αᵥβ₃ was confirmed, and compared with the targetability of cRGD-MNPs.

Each 1.0×10⁷ of PAE/KDR (integrin αᵥβ₃ overexpressing, test group) cells and A431 (integrin αᵥβ₃ low-expressing, control) cells were gathered, and washed with blocking buffer (0.2% FBS and 0.02% NaN3 in phosphate-buffered solution, pH 7.4, 10 mM) three times to decrease non-specific binding of the particles, Thereafter, the cells were treated with Apt_{αvβ3}-MNPs and cRGD-MNPs respectively, cultured at 4° C. for 2 hours, and then, washed to remove Apt_{αvβ3}-MNPs and cRGD-MNPs that were not supported in the cells.

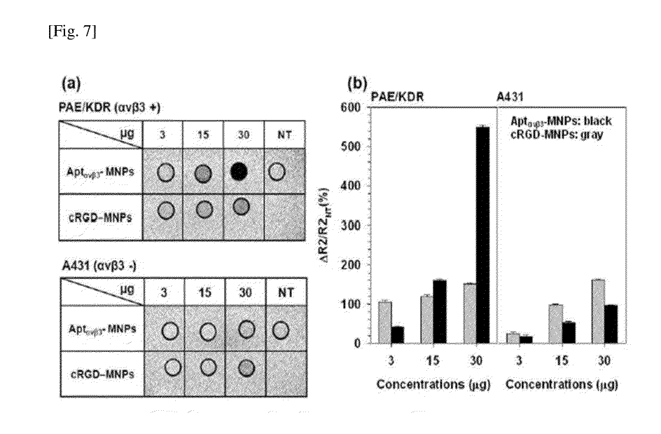

As shown in FIG. 7a, in the test group cells, although both Apt_{αvβ3}-MNPs and cRGD-MNPs exhibited targetability thus exhibiting dark contrast effect, at the same concentrations, Apt_{αvβ3}-MNPs exhibited higher targetability thus exhibiting darker contrast effect than the image of cRGD-MNPs. In the control cells, due to low Integrin_{•v•3} expression, neither Apt_{αvβ3}-MNPs and cRGD-MNPs exhibited contrast effect. In FIG. 7b, a graph of signal increase rate was plotted by measuring MR signal intensity (R2) from the image results of FIG. 7a, based on the R2 value of non-treated cells, and the same tendency as the image results of FIG. 7a is exhibited.

From the above results, it was confirmed that Apt_{αvβ3}-MNPs can be effectively targeted, and exhibits higher targetability than cRGD-MNPs.

3.5: Cancer Cell Imaging in an Animal Model Using Aptamer_{αvβ3}-Conjugated Magnetic Nanoparticles

In order to measure the targetability to cancer cells and distribution in cancer cells of the Apt_{αvβ3}-MNPs prepared in Example 2.4, an animal model prepared by transplanting 1.0×10⁷ A431 cells into 4-5 week aged male BALB/C-Slc nude mouse was used, 3T clinical MRI apparatus and micro-47 surface coil (Intera; Philips Medical Systems, Best, The Netherlands) were used for in vivo MR image, and the following parameters were used to obtain T2-weighted image: resolution of 234×234 mm, section thickness of 2.0 mm, TE=60 ms, TR=4000 ms, number of acquisitions=1.

MR images were measured before (pre-injection; Pre), immediately after (Imm), 1 hour, 3 hours, 24 hours after injection of Apt_{αvβ3}-MNPs and cRGD-MNPs of 200 μg Fe+Mn ion concentration into tail vein.

As shown in FIG. 8a, as the result of injecting Apt_{αvβ3}-MNPs, new blood vessels formed in cancer cell tissues showed very dark image contrast, and the image contrast was confirmed to appear more clearly through color mapping. It was also confirmed that the image contrast effect increased over time, and the darkest contrast appeared in 24 hour MR image. Thus, it was confirmed that Apt_{αvβ3}-MNPs can target integrin αᵥβ₃ that is expressed in new blood vessels of cancer tissues, and that it is accumulated in cancer tissues to effectively image cancer tissues.

As shown in FIG. 8a, as the result of injecting cRGD-MNPs, image contrast effect was exhibited in cancer cell tissues like Apt_{αvβ3}-MNPs, however, in case Apt_{αvβ3}-MNPs was injected, the image contrast effect continuously increased up to 24 hours, while in case cRGD-MNPs was injected, image contrast decreased 1 hour after the injection. Thus, it was confirmed that cRGD-MNPs has low integrin αᵥβ₃ targetability than Apt_{αvβ3}-MNPs.

In FIG. 8b, a graph of MR signal intensity (ΔR2/R2_{Pre}; ΔR2=R2−R2_{Pre}) of cancer tissues was plotted from the image results of FIG. 8a. As shown in the image results, in case Apt_{αvβ3}-MNPs was injected, signal intensity continuously increased up to 24 hours and increased maximum ˜33.7%, while in case cRGD-MNPs was injected, maximum ˜30.0% signal intensity appeared at 1 hour, and then, signal intensity decreased over time.

After injecting Apt_{αvβ3}-MNPs and cRGD-MNPs and confirming 24 hour images, each mouse was killed to extract cancer tissues, liver, brain, kidney and spleen, and the amounts of Apt_{αvβ3}-MNPs and cRGD-MNPs accumulated in each organ was measured by Inductively Coupled Plasma-Atom Emission Spectrometry (ICP-AES). Relative MNCs (Fe+Mn) concentration (ΔC/Cₛₐₗᵢₙₑ; ΔC=C−Cₛₐₗᵢₙₑ) of each organ was shown in FIG. 9.

As shown in FIG. 9, the concentration of Apt_{αvβ3}-MNPs (129±34.3%) accumulated in cancer tissues is much higher than that of cRGD-MNPs (17.6±15.4%), and thus, it was confirmed that Apt_{αvβ3}-MNPs more effectively targets integrin αᵥβ₃ expressed in cancer tissues.

1. Aptamer that specifically binds to integrin αᵥβ₃, comprises 5 to 20 modified bases that are substituted with a hydrophobic functional group selected from the group consisting of a naphthyl group, a benzyl group, a pyrrole benzyl group, and tryptophan at 5-position of dU (deoxyuracil), and has a total base length of 25 to 100.

2. The aptamer according to claim 1, wherein the aptamer comprises a base sequence selected from the group consisting of SEQ ID NO:1 to SEQ ID NO: 56, and has a total base length of 25 to 100.

3. The aptamer according to claim 2, wherein the aptamer further comprises a base sequence consisting of 3 to 25 bases at the 5′terminal, 3′terminal or both terminals of the base sequence selected from the group consisting of SEQ ID NO:1 to SEQ ID NO: 56, and has a total base length of 30 to 120.

4. The aptamer according to claim 3, wherein the based sequence that is further included at the 5′terminal, 3′terminal or both terminals is selected from the group consisting of SEQ ID NO: 57 to SEQ ID NO: 60.

5. The aptamer according to claim 4, wherein the aptamer has a base sequence of SEQ ID NO: 61 or SEQ ID NO: 62:
5′-TCAGCCGCCAGCCAGTTC-[N]-GACCAGAGCACCACAGAG-3′(SEQ ID NO: 61)
5′-AGTTC-[N]-GACCA-3′(SEQ ID NO: 62),
in the base sequence, ‘N’ consists of a base sequence selected from the group consisting of SEQ ID NO:1 to SEQ ID NO: 56, each base is independently selected from the group consisting of A, C, G, T, deoxy forms thereof, and modified bases that are substituted with a benzyl group at 5′-position of dU (deoxyuracil).

6. The aptamer according to claim 5, wherein the aptamer has a base sequence of SEQ ID NO: 63.

7. The aptamer according to claim 2, wherein the aptamer is modified by binding of at least one selected from the group consisting of idT (inverted deoxythymidine), LNA (Locked Nucleic Acid), 2′-methoxy nucleoside, 2′-amino nucleoside, 2′F-nucleoside, amine linker, thiol linker, and cholesterol at the 5′terminal, 3′terminal, or both terminals.

8. A composition for diagnosis of cancer or cancer metastasis, comprising the aptamer of claim 1 as an active ingredient.

9. The composition for diagnosis of cancer or cancer metastasis according to claim 8, wherein the cancer is at least one selected from the group consisting of skin cancer, prostate cancer, breast cancer, uterine cervical cancer, colorectal cancer, lung cancer, gallbladder cancer, pancreatic cancer and stomach cancer.

10. A composition for imaging tumor regions, comprising the aptamer of claim 1 as an active ingredient.

11. The composition according to claim 10, further comprising radio isotope, fluorophore, quantum dot, super paramagnetic particles or ultrasuper paramagnetic particles.

12. A nanoparticle contrast media comprising the composition for imaging tumor regions of claim 10.

13. A method for providing information for diagnosis on cancer or cancer metastasis comprising the steps of reacting a biological sample of a patient with the aptamer of claim 1, and measuring binding degree of aptamer in the biological sample,
wherein if the binding degree of aptamer in the biological sample of the patient is higher than that in a normal sample, the patient is determined as a cancer patient.


SEQUENCE LISTING (67 sequences)

SEQ ID NO: 1 - DNA, 40 bp, Artificial Sequence
  core sequence represented by Clone No. S003-A4-001-T7_A01, wherein n is BzdU [5-(N-Benzylaminocarbonylamide)-2′-deoxyuridine]
ncggaggcnnnacancggnaaccgagacnnaggacngnng

SEQ ID NO: 2 - DNA, 40 bp, Artificial Sequence
  core sequence represented by Clone No. S003-A4-002-T7_B01, wherein n is BzdU [5-(N-Benzylaminocarbonylamide)-2′-deoxyuridine]
ncncanncnnacacaaggccaganaaagngnagcaaagnn

SEQ ID NO: 3 - DNA, 40 bp, Artificial Sequence
  core sequence represented by Clone No. S003-A4-003-T7_C01, wherein n is BzdU [5-(N-Benzylaminocarbonylamide)-2′-deoxyuridine]
nagnngnacanncngagnnnagagcaaanaanagagncca

SEQ ID NO: 4 - DNA, 40 bp, Artificial Sequence
  core sequence represented by Clone No. S003-A4-004-T7_D01, wherein n is BzdU [5-(N-Benzylaminocarbonylamide)-2′-deoxyuridine]
nacnaaacacgcagacngaaannnnacancggnaacagnc

SEQ ID NO: 5 - DNA, 40 bp, Artificial Sequence
  core sequence represented by Clone No. S003-A4-005-T7_E01, wherein n is BzdU [5-(N-Benzylaminocarbonylamide)-2′-deoxyuridine]
nnnnaancnncncngncaganggcnggnagggngnangac

SEQ ID NO: 6 - DNA, 40 bp, Artificial Sequence
  core sequence represented by Clone No. S003-A4-006-T7_F01, wherein n is BzdU [5-(N-Benzylaminocarbonylamide)-2′-deoxyuridine]
cgagggagnnanggaganngngnngnnnaaggncggaacn

SEQ ID NO: 7 - DNA, 40 bp, Artificial Sequence
  core sequence represented by Clone No. S003-A4-007-T7_G01, wherein n is BzdU [5-(N-Benzylaminocarbonylamide)-2′-deoxyuridine]
cgagggagnnangggganngngnngnnnaaggncggaacn

SEQ ID NO: 8 - DNA, 40 bp, Artificial Sequence; identical to SEQ ID NO: 5
  core sequence represented by Clone No. S003-A4-008-T7_H01, wherein n is BzdU [5-(N-Benzylaminocarbonylamide)-2′-deoxyuridine]

SEQ ID NO: 9 - DNA, 41 bp, Artificial Sequence
  core sequence represented by Clone No. S003-A4-009-T7_A02, wherein n is BzdU [5-(N-Benzylaminocarbonylamide)-2′-deoxyuridine]
agnngccaannngcagccnagganacgnnnncgaaacngca

SEQ ID NO: 10 - DNA, 40 bp, Artificial Sequence
  core sequence represented by Clone No. S003-A4-010-T7_B02, wherein n is BzdU [5-(N-Benzylaminocarbonylamide)-2′-deoxyuridine]
ccgncagcgcggnncgaaggnacaannnnagancgcnaag

SEQ ID NO: 11 - DNA, 40 bp, Artificial Sequence; identical to SEQ ID NO: 5
  core sequence represented by Clone No. S003-A4-011-T7_C02, wherein n is BzdU [5-(N-Benzylaminocarbonylamide)-2′-deoxyuridine]

SEQ ID NO: 12 - DNA, 41 bp, Artificial Sequence; identical to SEQ ID NO: 9
  core sequence represented by Clone No. S003-A4-012-T7_D02, wherein n is BzdU [5-(N-Benzylaminocarbonylamide)-2′-deoxyuridine]

SEQ ID NO: 13 - DNA, 40 bp, Artificial Sequence; identical to SEQ ID NO: 5
  core sequence represented by Clone No. S003-A4-013-T7_E02, wherein n is BzdU [5-(N-Benzylaminocarbonylamide)-2′-deoxyuridine]

SEQ ID NO: 14 - DNA, 40 bp, Artificial Sequence
  core sequence represented by Clone No. S003-A4-014-T7_F02, wherein n is BzdU [5-(N-Benzylaminocarbonylamide)-2′-deoxyuridine]
nnnnaancnncncngncaganggcnggnagggngnannac

SEQ ID NO: 15 - DNA, 40 bp, Artificial Sequence
  core sequence represented by Clone No. S003-A4-015-T7_G02, wherein n is BzdU [5-(N-Benzylaminocarbonylamide)-2′-deoxyuridine]
acgnaaaggagacggannnngacccgngnanacncgacgc

SEQ ID NO: 16 - DNA, 39 bp, Artificial Sequence
  core sequence represented by Clone No. S003-A4-016-T7_H02, wherein n is BzdU [5-(N-Benzylaminocarbonylamide)-2′-deoxyuridine]
gannncggaanaaggccnnangaaccangagccngncnc

SEQ ID NO: 17 - DNA, 40 bp, Artificial Sequence
  core sequence represented by Clone No. S003-A4-017-T7_A03, wherein n is BzdU [5-(N-Benzylaminocarbonylamide)-2′-deoxyuridine]
ngnganangncnngnnaagcnncngangangcagggcngg

SEQ ID NO: 18 - DNA, 40 bp, Artificial Sequence
  core sequence represented by Clone No. S003-A4-018-T7_B03, wherein n is BzdU [5-(N-Benzylaminocarbonylamide)-2′-deoxyuridine]
ncnccnncnnaccccgngnagcaaaganncagcngaggag

SEQ ID NO: 19 - DNA, 40 bp, Artificial Sequence
  core sequence represented by Clone No. S003-A4-019-T7_C03, wherein n is BzdU [5-(N-Benzylaminocarbonylamide)-2′-deoxyuridine]
naanaagccacncggcgncacngnagnangnnnancnanc

SEQ ID NO: 20 - DNA, 40 bp, Artificial Sequence
  core sequence represented by Clone No. S003-A4-020-T7_D03, wherein n is BzdU [5-(N-Benzylaminocarbonylamide)-2′-deoxyuridine]
agcgngagacaggngngaggaggcaannnnacanaggnaa

SEQ ID NO: 21 - DNA, 40 bp, Artificial Sequence; identical to SEQ ID NO: 6
  core sequence represented by Clone No. S003-A4-021-T7_E03, wherein n is BzdU [5-(N-Benzylaminocarbonylamide)-2′-deoxyuridine]

SEQ ID NO: 22 - DNA, 41 bp, Artificial Sequence; identical to SEQ ID NO: 9
  core sequence represented by Clone No. S003-A4-022-T7_F03, wherein n is BzdU [5-(N-Benzylaminocarbonylamide)-2′-deoxyuridine]

SEQ ID NO: 23 - DNA, 41 bp, Artificial Sequence; identical to SEQ ID NO: 9
  core sequence represented by Clone No. S003-A4-023-T7_G03, wherein n is BzdU [5-(N-Benzylaminocarbonylamide)-2′-deoxyuridine]

SEQ ID NO: 24 - DNA, 40 bp, Artificial Sequence; identical to SEQ ID NO: 6
  core sequence represented by Clone No. S003-A4-024-T7_H03, wherein n is BzdU [5-(N-Benzylaminocarbonylamide)-2′-deoxyuridine]

SEQ ID NO: 25 - DNA, 40 bp, Artificial Sequence
  core sequence represented by Clone No. S003-A4-025-T7_A04, wherein n is BzdU [5-(N-Benzylaminocarbonylamide)-2′-deoxyuridine]
gnnnnaagaaannagcacaccgnngacnngnnnagnggcg

SEQ ID NO: 26 - DNA, 40 bp, Artificial Sequence
  core sequence represented by Clone No. S003-A4-026-T7_B04, wherein n is BzdU [5-(N-Benzylaminocarbonylamide)-2′-deoxyuridine]
gnnnnaagaaannagcacancgnngacnngnnnagnggcg

SEQ ID NO: 27 - DNA, 40 bp, Artificial Sequence
  core sequence represented by Clone No. S003-A4-027-T7_C04, wherein n is BzdU [5-(N-Benzylaminocarbonylamide)-2′-deoxyuridine]
aggncacnangannnngacccgngnnngcncgacgcgnaa

SEQ ID NO: 28 - DNA, 40 bp, Artificial Sequence
  core sequence represented by Clone No. S003-A4-028-T7_D04, wherein n is BzdU [5-(N-Benzylaminocarbonylamide)-2′-deoxyuridine]
aggncacnangannnngacccgngnnnacncgacgcgcaa

SEQ ID NO: 29 - DNA, 40 bp, Artificial Sequence
  core sequence represented by Clone No. S003-A4-029-T7_E04, wherein n is BzdU [5-(N-Benzylaminocarbonylamide)-2′-deoxyuridine]
ngnganangncnngnnaagcnncnganganaccgggcngg

SEQ ID NO: 30 - DNA, 40 bp, Artificial Sequence; identical to SEQ ID NO: 25
  core sequence represented by Clone No. S003-A4-030-T7_F04, wherein n is BzdU [5-(N-Benzylaminocarbonylamide)-2′-deoxyuridine]

SEQ ID NO: 31 - DNA, 40 bp, Artificial Sequence
  core sequence represented by Clone No. S003-A4-031-T7_G04, wherein n is BzdU [5-(N-Benzylaminocarbonylamide)-2′-deoxyuridine]
nnnnaancnncncngncaganggcnggnagggngnangaa

SEQ ID NO: 32 - DNA, 40 bp, Artificial Sequence
  core sequence represented by Clone No. S003-A4-032-T7_H04, wherein n is BzdU [5-(N-Benzylaminocarbonylamide)-2′-deoxyuridine]
nncagaccaannanggnaannncncaaancngagngncan

SEQ ID NO: 33 - DNA, 40 bp, Artificial Sequence
  core sequence represented by Clone No. S003-A4-033-T7_A05, wherein n is BzdU [5-(N-Benzylaminocarbonylamide)-2′-deoxyuridine]
ncgnannnngacccgngnanccncgangcggnnagcagca

SEQ ID NO: 34 - DNA, 40 bp, Artificial Sequence; identical to SEQ ID NO: 19
  core sequence represented by Clone No. S003-A4-034-T7_B05, wherein n is BzdU [5-(N-Benzylaminocarbonylamide)-2′-deoxyuridine]

SEQ ID NO: 35 - DNA, 40 bp, Artificial Sequence; identical to SEQ ID NO: 25
  core sequence represented by Clone No. S003-A4-035-T7_C05, wherein n is BzdU [5-(N-Benzylaminocarbonylamide)-2′-deoxyuridine]

SEQ ID NO: 36 - DNA, 40 bp, Artificial Sequence; identical to SEQ ID NO: 25
  core sequence represented by Clone No. S003-A4-036-T7_D05, wherein n is BzdU [5-(N-Benzylaminocarbonylamide)-2′-deoxyuridine]

SEQ ID NO: 37 - DNA, 40 bp, Artificial Sequence
  core sequence represented by Clone No. S003-A4-037-T7_E05, wherein n is BzdU [5-(N-Benzylaminocarbonylamide)-2′-deoxyuridine]
cgagggagnnangnaganngngnngnnnaaggncngaacn

SEQ ID NO: 38 - DNA, 40 bp, Artificial Sequence
  core sequence represented by Clone No. S003-A4-038-T7_F05, wherein n is BzdU [5-(N-Benzylaminocarbonylamide)-2′-deoxyuridine]
ncncanncnnacacaaggccagagaaagngnagcaaagnn

SEQ ID NO: 39 - DNA, 40 bp, Artificial Sequence
  core sequence represented by Clone No. S003-A4-039-T7_G05, wherein n is BzdU [5-(N-Benzylaminocarbonylamide)-2′-deoxyuridine]
gacnnnnacancggnaaagaacncaganangcacaagnna

SEQ ID NO: 40 - DNA, 40 bp, Artificial Sequence
  core sequence represented by Clone No. S003-A4-040-T7_H05, wherein n is BzdU [5-(N-Benzylaminocarbonylamide)-2′-deoxyuridine]
ncgagccanggncgagccccannnnacancggnaagggcn

SEQ ID NO: 41 - DNA, 40 bp, Artificial Sequence; identical to SEQ ID NO: 25
  core sequence represented by Clone No. S003-A4-041-T7_A06, wherein n is BzdU [5-(N-Benzylaminocarbonylamide)-2′-deoxyuridine]

SEQ ID NO: 42 - DNA, 40 bp, Artificial Sequence; identical to SEQ ID NO: 17
  core sequence represented by Clone No. S003-A4-042-T7_B06, wherein n is BzdU [5-(N-Benzylaminocarbonylamide)-2′-deoxyuridine]

SEQ ID NO: 43 - DNA, 40 bp, Artificial Sequence; identical to SEQ ID NO: 6
  core sequence represented by Clone No. S003-A4-043-T7_C06, wherein n is BzdU [5-(N-Benzylaminocarbonylamide)-2′-deoxyuridine]

SEQ ID NO: 44 - DNA, 41 bp, Artificial Sequence
  core sequence represented by Clone No. S003-A4-044-T7_D06, wherein n is BzdU [5-(N-Benzylaminocarbonylamide)-2′-deoxyuridine]
agcnaacgacannnnacancggnaagncaaaccncagcacn

SEQ ID NO: 45 - DNA, 46 bp, Artificial Sequence
  core sequence represented by Clone No. S003-A4-045-T7_E06, wherein n is BzdU [5-(N-Benzylaminocarbonylamide)-2′-deoxyuridine]
naancnggnagnnnaagcacanngnganngcacgcggangnnngan

SEQ ID NO: 46 - DNA, 39 bp, Artificial Sequence; identical to SEQ ID NO: 16
  core sequence represented by Clone No. S003-A4-046-T7_F06, wherein n is BzdU [5-(N-Benzylaminocarbonylamide)-2′-deoxyuridine]

SEQ ID NO: 47 - DNA, 41 bp, Artificial Sequence; identical to SEQ ID NO: 9
  core sequence represented by Clone No. S003-A4-047-T7_G06, wherein n is BzdU [5-(N-Benzylaminocarbonylamide)-2′-deoxyuridine]

SEQ ID NO: 48 - DNA, 40 bp, Artificial Sequence
  core sequence represented by Clone No. S003-A4-048-T7_H06, wherein n is BzdU [5-(N-Benzylaminocarbonylamide)-2′-deoxyuridine]
ncnggcaacnnnnacancggnaagccnanngagcgcgacn

SEQ ID NO: 49 - DNA, 40 bp, Artificial Sequence
  core sequence represented by Clone No. S003-A4-049-T7_A07, wherein n is BzdU [5-(N-Benzylaminocarbonylamide)-2′-deoxyuridine]
nnnnaancnncncngncaganggcnggnagggngngngac

SEQ ID NO: 50 - DNA, 40 bp, Artificial Sequence; identical to SEQ ID NO: 6
  core sequence represented by Clone No. S003-A4-050-T7_B07, wherein n is BzdU [5-(N-Benzylaminocarbonylamide)-2′-deoxyuridine]

SEQ ID NO: 51 - DNA, 40 bp, Artificial Sequence
  core sequence represented by Clone No. S003-A4-051-T7_C07, wherein n is BzdU [5-(N-Benzylaminocarbonylamide)-2′-deoxyuridine]
nanngggaggngggggccannnacanaggnaacagccacn

SEQ ID NO: 52 - DNA, 40 bp, Artificial Sequence; identical to SEQ ID NO: 5
  core sequence represented by Clone No. S003-A4-052-T7_D07, wherein n is BzdU [5-(N-Benzylaminocarbonylamide)-2′-deoxyuridine]

SEQ ID NO: 53 - DNA, 41 bp, Artificial Sequence
  core sequence represented by Clone No. S003-A4-053-T7_E07, wherein n is BzdU [5-(N-Benzylaminocarbonylamide)-2′-deoxyuridine]
agnngncaannngcagccnanganacgnnnncgaaacngca

SEQ ID NO: 54 - DNA, 41 bp, Artificial Sequence
  core sequence represented by Clone No. S003-A4-054-T7_F07, wherein n is BzdU [5-(N-Benzylaminocarbonylamide)-2′-deoxyuridine]
cgaacggaanggancagnccngggcaannnnacanaggnaa

SEQ ID NO: 55 - DNA, 40 bp, Artificial Sequence; identical to SEQ ID NO: 6
  core sequence represented by Clone No. S003-A4-055-T7_G07, wherein n is BzdU [5-(N-Benzylaminocarbonylamide)-2′-deoxyuridine]

SEQ ID NO: 56 - DNA, 40 bp, Artificial Sequence
  core sequence represented by Clone No. S003-A4-056-T7_H07, wherein n is BzdU [5-(N-Benzylaminocarbonylamide)-2′-deoxyuridine]
aggcnagcgggacagnannngaaccgngnanccncgacgc

SEQ ID NO: 57 - DNA, 18 bp, Artificial Sequence
  5′-end sequence of aptamer
tcagccgccagccagttc

SEQ ID NO: 58 - DNA, 18 bp, Artificial Sequence
  3′-end sequence of aptamer
gaccagagcaccacagag

SEQ ID NO: 59 - DNA, 5 bp, Artificial Sequence
  5′-end sequence of aptamer
agttc

SEQ ID NO: 60 - DNA, 5 bp, Artificial Sequence
  3′-end sequence of aptamer
gacca

SEQ ID NO: 61 - DNA, 37 bp, Artificial Sequence
  aptamer sequence
tcagccgccagccagttcngaccagagcaccacagag

SEQ ID NO: 62 - DNA, 11 bp, Artificial Sequence
  aptamer sequence
agttcngacca

SEQ ID NO: 63 - DNA, 50 bp, Artificial Sequence
  aptamer HO-2100-25-2
agttcgnnnnaagaaannagcacaccgnngacnngnnnagnggcggacca

SEQ ID NO: 64 - DNA, 76 bp, Artificial Sequence
  antisense library, wherein n is any one selected from consiting of A, G, C, T, and BzdU
ctctgtggtgctctggtcnnnnnnnnnnnnnnnnnnnnnnnnnnnnnnnnnnnnnnnngaactggctggcggctga

SEQ ID NO: 65 - DNA, 18 bp, Artificial Sequence
  5′ primer
tcagccgccagccagttc

SEQ ID NO: 66 - DNA, 18 bp, Artificial Sequence
  3′ primer
ctctgtggtgctctggtc

SEQ ID NO: 67 - DNA, 17 bp, Artificial Sequence
  M13 primer
caggaaacagctatgac